(19) United States
(12) Patent Application Publication
(10) Pub. No.: US 2022/0071562 A1
(43) Pub. Date: Mar. 10, 2022

(54) FACE MASK WITH INTEGRATED PHYSIOLOGICAL SENSORS

(71) Applicant: Masimo Corporation, Irvine, CA (US)

(72) Inventors: Massi Joe E. Kiani, Laguna Niguel, CA (US); Naoki Kokawa, Irvine, CA (US)

(21) Appl. No.: 17/462,766

(22) Filed: Aug. 31, 2021

Related U.S. Application Data
(60) Provisional application No. 63/091,789, filed on Oct. 14, 2020.
(60) Provisional application No. 63/075,754, filed on Sep. 8, 2020.

(51) Int. Cl. A61B 5/00; A61B 5/0205; A61B 5/1455; A62B 23/02

(52) U.S. Cl. CPC A61B 5/6803; A61B 5/02055; A61B 5/14552; A61B 5/02438; A61B 5/742; A61B 5/0002; A62B 23/025

(57) ABSTRACT

A face mask including a body portion configured to be secured to a user's face and cover a mouth and nasal passages of the user, a processor, and an oximetry sensor operably positioned at the user's nose when the body portion is secured to the user's face. In some implementations, the oximetry sensor includes at least one emitter configured to emit light into tissue of the user's nose and at least one detector configured to detect at least a portion of the emitted light after passing through the tissue and transmit one or more signals to the processor responsive to detected light. In some implementations, the processor determines one or more physiological parameters based on the one or more signals transmitted by said at least one detector. In some implementations, the face mask is configured to wirelessly transmit the one or more physiological parameters to a mobile computing device.

### INCORPORATION BY REFERENCE TO ANY PRIORITY APPLICATIONS

This application claims the benefit of priority under 35 U.S.C. § 119(e) to U.S. Patent Application No. 63/075,754, entitled “Face Mask with Integrated Physiological Sensors”, filed Sep. 8, 2020, and U.S. Patent Application No. 63/091,789, entitled “Face Mask with Integrated Physiological Sensors”, filed Oct. 14, 2020. All of the above-mentioned applications are hereby incorporated by reference herein in their entireties. Any and all applications for which a foreign or domestic priority claim is identified in the Application Data Sheet as filed with the present application are hereby incorporated by reference under 37 CFR 1.57.

### TECHNICAL FIELD

The present disclosure relates to face masks with integrated physiological sensors for measuring one or more physiological parameters of a user.

### BACKGROUND

Face masks, such as disposable surgical masks, are often employed to protect a wearer from exposure to air pollutants or airborne particulates that may be associated with infections. Some face masks cover the wearer's mouth and nose and have straps that secure the face mask in place. Recent trends of emerging infections have created increased worldwide demand for face masks in order to reduce the likelihood of transmission.

### SUMMARY

There is a growing need for face masks that comfortably secure to a wearer's face, provide filtration of ambient air and/or exhaled gas, and monitor the wearer's physiological information. Disclosed herein are various embodiments of face masks that provide filtration of particulates from ambient air and/or exhaled gas and that include one or more physiological sensors which can provide a variety of information useful to monitor a wearer's physiological status.

Disclosed herein is a face mask configured to secure to a face of a user, provide filtration of air prior to inhalation by the user, and measure one or more physiological parameters of the user, the face mask comprising: a body portion configured to be secured to the user's face and cover a mouth and nasal passages of the user; at least one strap connected to the body portion and configured to secure the body portion to the user; a power source; one or more hardware processors; and an oximetry sensor in communication with said one or more hardware processors and configured to be positioned at the user's nose when the face mask is in use. The body portion can be configured to at least partially define an interior space when secured to the user's face and can comprise: an upper section configured to be positioned around at least a portion of a nose of the user and conform to at least a portion of a shape of the user's nose when the body portion is secured to the user's face; a lower section configured to be positioned proximate a chin of the user when the body portion is secured to the user's face; an inlet configured to allow air to flow into said interior space during inhalation by the user; an outlet configured to allow exhaled gases from the user to flow outside said interior space, wherein said inlet and outlet are located in the lower section of the body portion and are configured to face downward when the body portion is secured to the user's face; and a filter positioned adjacent the inlet and the outlet, wherein the filter is configured to filter out particles in said air prior to inhalation by the user. The oximetry sensor can comprise at least one emitter configured to emit one or more wavelengths into tissue of the user's nose and at least one detector configured to detect at least a portion of the emitted light after passing through at least a portion of said tissue, said at least one detector configured to transmit one or more signals to said one or more hardware processors responsive to detected light. The one or more hardware processors can be configured to determine said one or more physiological parameters based on said one or more signals transmitted by said at least one detector.

In some implementations, said one or more physiological parameters comprises at least one of oxygen saturation and pulse rate. In some implementations, the oximetry sensor is configured to secure around at least a portion of a nostril of the user. In some implementations, the oximetry sensor comprises a clip. In some implementations, the at least one emitter and the at least one detector are arranged in a transmissive arrangement. In some implementations, the at least one emitter and the at least one detector are arranged in a reflectance arrangement. In some implementations, the oximetry sensor is secured to the upper section of the body portion such that, when the upper section is positioned around the at least the portion of the nose of the user, the oximetry sensor is positioned adjacent skin of the nose and the at least one emitter and the at least one detector are arranged in a reflectance arrangement with respect to the skin.

In some implementations, the face mask further comprises a circuit board coupled to the oximetry sensor via a cable. In some implementations, said power source comprises a battery. In some implementations, said battery is rechargeable. In some implementations, said battery is not rechargeable. In some implementations, said battery is positioned within the lower section of said body portion. In some implementations, the lower section of said body portion is configured to allow said battery to be replaced. In some implementations, said at least one strap comprises a first strap configured to wrap around a portion of the user's head above ears of the user and a second strap configured to wrap around a portion of the user's head below the ears. In some implementations, the face mask further comprises a temperature sensor operably positioned by the upper section of the body portion such that, when the upper section is positioned around the at least the portion of the nose of the user, the temperature sensor is positioned adjacent skin of the nose. In some implementations, said inlet and said outlet occupy the same space in the body portion. In some implementations, said filter is further configured to filter out particles in said exhaled gases prior to exiting said interior space.

In some implementations, the face mask further comprises a status indicator configured to indicate at least one of a status of the face mask and a status of the user. In some implementations, said status indicator comprises one or more light sources. In some implementations, said one or more hardware processors are configured to alter a characteristic of said one or more light sources based on said determined one or more physiological parameters. In some implementations, said one or more hardware processors are configured to alter said characteristic of said one or more light sources based on a comparison of said determined one or more physiological parameters to one or more thresholds. In some implementations, said one or more hardware processors are configured to alter a color of said one or more light sources based on said comparison. In some implementations, said one or more hardware processors are configured to cause said one or more light sources to blink based on said comparison. In some implementations, said face mask is configured to wirelessly transmit said determined one or more physiological parameters to a mobile computing device. In some implementations, said face mask is configured to wirelessly transmit said determined one or more physiological parameters to said mobile computing device over a Bluetooth® wireless protocol.

In some implementations, a system comprises any of the face masks described above and a mobile software application configured to execution by one or more hardware processors of said mobile computing device, wherein the mobile software application is configured to execute commands to enable the mobile computing device to: wirelessly receive said determined one or more physiological parameters; generate a graphical user interface on a display of the mobile computing device; and display, in at least a portion of the graphical user interface, at least one of said determined one or more physiological parameters and information related to said determined one or more physiological parameters. In some implementations, the mobile software application is further configured to execute commands to enable the mobile computing device to wirelessly transmit, to a remote monitoring system, said at least one of said determined one or more physiological parameters and information related to said determined one or more physiological parameters. In some implementations, said mobile computing device comprises a mobile phone.

Disclosed herein is a face mask configured to secure to a face of a user and measure one or more physiological parameters of the user, the face mask comprising: a body portion configured to be secured to the user's face and cover a mouth and nasal passages of the user; at least one strap connected to the body portion and configured to secure the body portion to the user; a power source; one or more hardware processors; and an oximetry sensor in communication with said one or more hardware processors and configured to be positioned at the user's nose when face mask is in use, wherein the oximetry sensor comprises at least one emitter configured to emit one or more wavelengths into tissue of the user's nose and at least one detector configured to detect at least a portion of the emitted light after passing through at least a portion of said tissue, said at least one detector configured to transmit one or more signals to said one or more hardware processors responsive to detected light. The one or more hardware processors can be configured to determine said one or more physiological parameters based on said one or more signals transmitted by said at least one detector.

In some implementations, the body portion is configured to at least partially define an interior space when secured to the user's face. In some implementations, the body portion comprises: an upper section configured to be positioned around at least a portion of a nose of the user and conform to at least a portion of a shape of the user's nose when the body portion is secured to the user's face; a lower section configured to be positioned near a chin of the user when the body portion is secured to the user's face; an inlet configured to allow air to flow into said interior space during inhalation by the user; an outlet configured to allow exhaled gases from the user to flow outside said interior space; and a filter positioned adjacent the inlet and the outlet, wherein the filter is configured to filter out particles in said air prior to inhalation by the user. In some implementations, said inlet and outlet are located in the lower section and are configured to face downward when the body portion is secured to the user's face. In some implementations, said inlet and said outlet occupy the same space in the body portion. In some implementations, the lower section of said body portion comprises: an outer wall that faces downward when the body portion is secured to the user's face; an inner wall spaced above the outer wall; and a cavity positioned between the outer and inner walls, where said filter is positioned within said cavity. In some implementations, the face mask further comprises a first plurality of openings in said outer wall and a second plurality of openings in said inner wall, wherein said inlet and said outlet are at least partially defined by said first and second plurality of openings. In some implementations, each of said first plurality of openings comprises a vent having a linear shape and wherein each of said second plurality of openings comprises a hole having circular shape.

In some implementations, the oximetry sensor is operably positioned such that, when the upper section is positioned around the at least the portion of the nose of the user, the oximetry sensor is positioned adjacent skin of the nose and the at least one emitter and the at least one detector are arranged in a reflectance arrangement with respect to the skin. In some implementations, the face mask further comprises a temperature sensor operably positioned such that, when the upper section is positioned around the at least the portion of the nose of the user, the temperature sensor is positioned adjacent skin of the nose. In some implementations, said one or more physiological parameters comprises at least one of oxygen saturation and pulse rate.

In some implementations, the face mask further comprises a status indicator configured to indicate at least one of a status of the face mask and a status of the user. In some implementations, said status indicator comprises one or more light sources. In some implementations, said status indicator comprises an LED. In some implementations, said one or more hardware processors are configured to alter a characteristic of said one or more light sources based on said determined one or more physiological parameters. In some implementations, said one or more hardware processors are configured to alter said characteristic of said one or more light sources based on a comparison of said determined one or more physiological parameters to one or more thresholds. In some implementations, said one or more hardware processors are configured to alter a color of said one or more light sources based on said comparison. In some implementations, said one or more hardware processors are configured to cause said one or more light sources to blink based on said comparison. In some implementations, said face mask is configured to wirelessly transmit said determined one or more physiological parameters to a mobile computing device. In some implementations, said face mask is configured to wirelessly transmit said determined one or more physiological parameters to said mobile computing device over a Bluetooth® wireless protocol.

In some implementations, a system comprises any of the face masks described above and a mobile software application configured to execution by one or more hardware processors of said mobile computing device, wherein the mobile software application is configured to execute commands to enable the mobile computing device to: wirelessly receive said determined one or more physiological parameters; generate a graphical user interface on a display of the mobile computing device; and display, in at least a portion of the graphical user interface, at least one of said determined one or more physiological parameters and information related to said determined one or more physiological parameters. In some implementations, the mobile software application is further configured to execute commands to enable the mobile computing device to wirelessly transmit, to a remote monitoring system, said at least one of said determined one or more physiological parameters and information related to said determined one or more physiological parameters. In some implementations, said mobile computing device comprises a mobile phone.

Disclosed herein is a face mask configured to be secured to a user and measure one or more physiological parameters of the user, the face mask comprising: a body portion configured to be secured to the user's face and cover a mouth and nasal passages of the user, wherein the body portion is configured to at least partially define an interior space when secured to the user's face, and wherein the body portion comprises an outlet configured to allow exhaled gas from the user to flow outside said interior space; at least one strap connected to the body portion and configured to secure the body portion to the user's face; a power source; a first temperature sensor operably positioned by the body portion adjacent skin of the nose when the body portion is secured to the user's face, said first temperature sensor usable to measure skin temperature at a nose of the user when in use; a second temperature sensor operably positioned in an exit path of the exhaled gas from the user flowing out of said interior space through the outlet, said second temperature sensor usable to measure temperature of said exhaled gas; and one or more hardware processors configured to determine a status of the user based on skin temperature measured using said first temperature sensor and exhaled gas temperature measured using said second temperature sensor.

In some implementations, the body portion comprises: an upper section configured to be positioned around at least a portion of a nose of the user and conform to at least a portion of a shape of the user's nose when the body portion is secured to the user's face, wherein the first temperature sensor is operably positioned by the upper section of the body portion to be adjacent to the skin of the user's nose; and a lower section configured to be positioned near a chin of the user when the body portion is secured to the user's face, wherein the outlet is located in the lower section and is configured to face downward when the body portion is secured to the user's face. In some implementations, the body portion comprises: an inlet configured to allow air to flow into said interior space during inhalation by the user, wherein the inlet is located in the lower section of the body portion; and a filter positioned adjacent the inlet and the outlet, wherein the filter is configured to filter out particles in said air prior to inhalation by the user. In some implementations, said inlet and said outlet occupy the same space in the lower section of said body portion. In some implementations, said filter is further configured to filter out particles in said exhaled gases prior to exiting said interior space. In some implementations, the lower section of the body portion comprises a cavity, and wherein said filter is positioned within said cavity. In some implementations, the lower section of said body portion comprises: an outer wall that faces downward when the body portion is secured to the user's face; and an inner wall spaced above the outer wall. In some implementations, said cavity is positioned between the outer and inner walls. In some implementations, the face mask further comprises a first plurality of openings in said outer wall and a second plurality of openings in said inner wall, wherein said inlet and said outlet are at least partially defined by said first and second plurality of openings. In some implementations, each of said first plurality of openings comprises a vent having a linear shape and wherein each of said second plurality of openings comprises a hole having circular shape. In some implementations, said second temperature sensor is positioned atop said filter within said cavity. In some implementations, said second temperature sensor is secured to said filter within said cavity. In some implementations, said filter comprises a corrugated structure.

In some implementations, the face mask further comprises a status indicator configured to provide a visual indication related to said status. In some implementations, said status indicator comprises one or more light sources. In some implementations, said one or more hardware processors are configured to alter a characteristic of said one or more light sources based on said status. In some implementations, said one or more hardware processors are configured to cause said one or more light sources to change color based on said status. In some implementations, said one or more hardware processors are configured to cause said one or more light sources to blink based on said status. In some implementations, said face mask is configured to wirelessly transmit, to a mobile computing device, at least one of said skin temperature measurements, said temperature of said exhaled gases, and said status.

In some implementations, said face mask is configured to communicate with said mobile computing device via a Bluetooth® wireless protocol. In some implementations, a system comprises any of the face masks described above and a mobile software application configured to execution by one or more hardware processors of said mobile computing device, wherein the mobile software application is configured to execute commands to enable the mobile computing device to: wirelessly receive said at least one of said skin temperature measurements, said temperature of said exhaled gases, and said status; generate a graphical user interface on a display of the mobile computing device; and display, in at least a portion of the graphical user interface, said at least one of said skin temperature measurements, said temperature of said exhaled gases, and said status. In some implementations, said mobile computing device comprises a mobile phone. In some implementations, a system comprises any of the face masks described above and said determining said status of the user comprises determining whether the user has a fever.

Disclosed herein is a face mask configured to secure to a user and measure one or more physiological parameters of the user, the face mask comprising: a body portion configured to be secured to the user's face and cover a mouth and nasal passages of the user, wherein the body portion is configured to at least partially define an interior space when secured to the user's face, and wherein the body portion comprises an outlet configured to allow exhaled gas from the user to flow outside said interior space; at least one strap connected to the body portion and configured to secure the body portion to the user; a power source; a temperature sensor operably positioned in an exit path of the exhaled gas from the user flowing out of said interior space through the outlet, said temperature sensor usable to measure temperature of said exhaled gas; and one or more hardware processors configured to determine a status of the user based on said temperature of said exhaled gases received from said second temperature sensor.

In some implementations, the body portion comprises: an upper section configured to be positioned around at least a portion of a nose of the user and conform to at least a portion of a shape of the user's nose when the body portion is secured to the user's face; and a lower section configured to be positioned near a chin of the user when the body portion is secured to the user's face, wherein the outlet is located in the lower section and is configured to face downward when the body portion is secured to the user's face. In some implementations, the body portion further comprises: an inlet configured to allow air to flow into said interior space during inhalation by the user, wherein the inlet is located in the lower section of the body portion; and a filter positioned adjacent the inlet and the outlet, wherein the filter is configured to filter out particles in said air prior to inhalation by the user. In some implementations, said inlet and said outlet are defined by a same portion of the lower section of said body portion. In some implementations, said filter is further configured to filter out particles in said exhaled gases prior to exiting said interior space. In some implementations, the lower section of the body portion comprises a cavity, and wherein said filter is positioned within said cavity. In some implementations, said temperature sensor is positioned atop said filter. In some implementations, said temperature sensor is secured to said filter. In some implementations, said filter comprises a corrugated structure. In some implementations, the face mask further comprises a status indicator configured to provide a visual indication related to said status. In some implementations, said status indicator comprises one or more light sources. In some implementations, said one or more hardware processors are configured to alter a characteristic of said one or more light sources based on said status. In some implementations, said one or more hardware processors are configured to cause said one or more light sources to change color based on said status. In some implementations, said one or more hardware processors are configured to cause said one or more light sources to blink based on said status. In some implementations, said face mask is configured to wirelessly transmit, to a mobile computing device, at least one of said temperature of said exhaled gases and said status.

In some implementations, a system comprises any of the face masks described above and a mobile software application configured to execution by one or more hardware processors of said mobile computing device, wherein the mobile software application is configured to execute commands to enable the mobile computing device to: wirelessly receive said at least one of said temperature of said exhaled gases and said status; generate a graphical user interface on a display of the mobile computing device; and display, in at least a portion of the graphical user interface, said at least one of said temperature of said exhaled gases and said status. In some implementations, said mobile computing device comprises a mobile phone. In some implementations, said face mask is configured to communicate with said mobile computing device via a Bluetooth® wireless protocol. In some implementations, said determining said status of the user comprises determining whether the user has a fever.

Disclosed herein is a face mask configured to be secured to a user and measure one or more physiological parameters of the user, the face mask comprising: a body portion; at least one strap, and a capnography module. The body portion can be secured to the user's face and cover a mouth and nasal passages of the user and/or the body portion can at least partially define an interior space when secured to the user's face. The body portion can comprise: an upper section configured to be positioned around at least a portion of a nose of the user and conform to at least a portion of a shape of the user's nose when the body portion is secured to the user's face; a lower section configured to be positioned near a chin of the user when the body portion is secured to the user's face; an outlet configured to allow exhaled gases from the user to flow outside said interior space, wherein said outlet is located in the lower section and is configured to face downward when the body portion is secured to the user's face. The at least one strap can be connected to the body portion and configured to secure the body portion to the user. The capnography module can be connected to the lower section of the body portion adjacent said outlet, the capnography module usable for determining one or more physiological parameters based on said exhaled gases of the user.

In some implementations, said capnography module is usable for determining at least one of an end-tidal carbon dioxide (EtCO₂) and respiration rate of the user. In some implementations, said capnography module is configured to removably connect to the lower section of the body portion adjacent said outlet. In some implementations, the body portion further comprises: an inlet configured to allow air to flow into said interior space during inhalation by the user, wherein the inlet is located in the lower section of the body portion; and a filter positioned adjacent the inlet and the outlet, wherein the filter is configured to filter out particles in said air prior to inhalation by the user. In some implementations, said inlet and said outlet are defined by a same portion of the lower section of said body portion. In some implementations, said filter is further configured to filter out particles in said exhaled gases prior to exiting said interior space. In some implementations, the lower section of the body portion comprises a cavity, and wherein said filter is positioned within said cavity. In some implementations, the lower section of said body portion comprises: an outer wall that faces downward when the body portion is secured to the user's face; and an inner wall spaced above the outer wall. In some implementations, said cavity is positioned between the outer and inner walls. In some implementations, the face mask further comprises a first plurality of openings in said outer wall and a second plurality of openings in said inner wall, wherein said inlet and said outlet are at least partially defined by said first and second plurality of openings. In some implementations, each of said first plurality of openings comprises a vent having a linear shape and wherein each of said second plurality of openings comprises a hole having circular shape. In some implementations, said filter comprises: a first frame and a second frame; a wicking element positioned between the first and second frames and configured to wick away moisture from said exhaled gases; and a filtration element positioned between the first and second frames and configured to filter out said particles in said air prior to inhalation by the user.

In some implementations, the face mask further comprises: a power source, one or more hardware processors, and a status indicator configured to indicate a status of the user based on said determined one or more physiological parameters. In some implementations, said status indicator comprises one or more light sources. In some implementations, said one or more hardware processors are configured to alter a characteristic of said one or more light sources based on said status. In some implementations, said one or more hardware processors are configured to cause said one or more light sources to change color based on said status. In some implementations, said one or more hardware processors are configured to cause said one or more light sources to blink based on said status. In some implementations, said one or more hardware processors are configured to determine said status by comparing said determined one or more physiological parameters to one or more thresholds. In some implementations, said face mask is configured to wirelessly transmit, to a mobile computing device, said determined one or more physiological parameters.

In some implementations, a system comprises any of the face masks described above and a mobile software application configured to execution by one or more hardware processors of said mobile computing device, wherein the mobile software application is configured to execute commands to enable the mobile computing device to: wirelessly receive said determined one or more physiological parameters; generate a graphical user interface on a display of the mobile computing device; and display, in at least a portion of the graphical user interface, at least one of said determined one or more physiological parameters and information related to said determined one or more physiological parameters. In some implementations, said face mask is configured to communicate with said mobile computing device via a Bluetooth® wireless protocol. In some implementations, said mobile computing device comprises a mobile phone.

At least some aspects of the present disclosure provide a face mask configured to secure to a face of a user and measure one or more physiological parameters of the user. The face mask can include: a body portion configured to cover a mouth and nasal passages of the user, a pulse oximetry sensor coupled to the body portion, a processor, and at least one temperature sensor coupled to the body portion, wherein the at least one temperature sensor is configured to determine a temperature of the user. The pulse oximetry sensor can include an emitter configured to transmit light of one or more wavelengths into tissue of the user and a detector configured to detect light attenuated by the tissue of the user and generate at least one signal based on the detected light. The processor can be configured to determine a measurement of the one or more physiological parameters based on the generated at least one signal.

In some embodiments, the face mask further comprises at least one strap coupled to the body portion and configured to secure around an ear of the user. In some embodiments, the body portion is configured to secure to skin of the user's face. In some embodiments, the body portion comprises an adhesive material configured to allow the body portion to adhere to the skin of the user's face. In some embodiments, the face mask further comprises a circuit board coupled with the body portion, wherein the circuit board comprises the processor. In some embodiments, the circuit board is positioned in an electronics module of the body portion. In some embodiments, the pulse oximetry sensor comprises the processor. In some embodiments, the pulse oximetry sensor is configured to secure to a portion of the nose of the user. In some embodiments, the pulse oximetry sensor is configured to secure to a portion of an ear of the user. In some embodiments, the emitter and detector of the pulse oximetry sensor are arranged in a reflectance measurement configuration.

In some embodiments, the at least one temperature sensor comprises a first temperature sensor and a second temperature sensor, the first and second temperature sensors coupled with the body portion and spaced away from one another, wherein the first temperature sensor is configured to measure a body temperature based on thermal energy from skin of the user's face and the second temperature sensor is configured to measure an ambient temperature. In some embodiments, the body portion of the face mask comprises a moisture wicking material configured to transport moisture from an interior space defined between the face mask and the user's face to an exterior surface of the body portion facing away from the user's face during use.

At least some aspects of the present disclosure provide a face mask configured to secure to a face of a user, the face mask comprising: a body portion configured to cover a mouth and nasal passages of the user, the body portion having a first surface configured to face toward the user when the face mask is in use and a second surface opposite the first surface and configured to face away from the user's face when the face mask is in use; a display connected to the body portion along the second surface; one or more cameras connected to the body portion along the first surface and configured to face toward the user's face when the face mask is in use, the one or more cameras configured to capture one or more images of at least a portion of the user's face; one or more hardware processors operatively connected to the one or more cameras. The one or more hardware processors can be configured to: receive the captured one or more images from the one or more cameras; determine a facial expression of the user based on the received, captured one or more images; and generate a visual representation of the facial expression on the display based on the determined facial expression.

In some embodiments, the face mask further comprises one or more sensors configured to generate one or more signals responsive to motion of the at least the portion of the user's face. The one or more hardware processors can be operatively connected to the one or more sensors and configured to receive said one or more signals from the one or more sensors. The one or more hardware processors can be further configured to determine the facial expression of the user based on said received one or more signals. In some embodiments, the one or more sensors comprises an accelerometer. In some embodiments, the one or more sensors comprises a gyroscope. In some embodiments, the one or more sensors comprises an acoustic sensor. In some embodiments, the one or more cameras comprises: a first camera configured to capture one or more images of a first portion of the user's face proximate to a right corner of the user's mouth; a second camera configured to capture one or more images of a second portion of the user's face proximate to a left corner of the user's mouth; and a third camera configured to capture one or more images of the user's mouth. In some embodiments, the one or more hardware processors are configured to determine the facial expression of the user based on the received, captured one or more images at least by comparing the one or more images with one or more stored reference images. In some embodiments, the display is a flexible display.

At least some aspects of the present disclosure provide a face mask configured to secure to a face of a user, the face mask comprising: a body portion configured to cover a mouth and nasal passages of the user, the body portion having a first surface configured to face toward the user's face when the face mask is in use and a second surface opposite the first surface and configured to face away from the user's face when the face mask is in use; a display connected to the body portion along the second surface; one or more cameras connected to the body portion along the first surface and configured to face toward the user when the face mask is in use, the one or more cameras configured to capture one or more images of at least a portion of the user's face; and one or more hardware processors operatively connected to the one or more cameras and the display, the one or more hardware processors configured to receive the captured one or more images from the one or more cameras and present, on the display, said received, captured one or more images.

At least some aspects of the present disclosure provide a face mask configured to secure to a face of a user, the face mask comprising: a body portion configured to cover a mouth and nasal passages of the user, the body portion having a first surface configured to face toward the user's face when the face mask is in use and a second surface opposite the first surface and configured to face away from the user's face when the face mask is in use; and a cannula comprising one or more prongs configured to secure to one or more nasal passages of the user, wherein the cannula is operatively connected to the body portion and configured to direct exhaled air from the user's nasal passages away from the user's nostrils when the face mask is in use. In some embodiments, the cannula comprises two prongs, each of the two prongs configured to be received within a respective one of the user's nostrils. In some embodiments, the two prongs extend through the body portion and are configured to direct the exhaled air from the user's nostrils outside an interior space defined between the body portion and the user's face when the face mask is in use. In some embodiments, the one or more prongs are configured to extend from the user's nostrils to a portion of the body portion configured to be positioned proximate a chin of the user when the face mask is in use. In some embodiments, the one or more prongs extend along and are secured to the first surface of the body portion.

At least some aspects of the present disclosure provide a face mask configured to secure to a face of a user, the face mask comprising: a body portion configured to cover a mouth and nasal passages of the user, the body portion having a first surface configured to face toward the user's face when the face mask is in use and a second surface opposite the first surface and configured to face away from the user's face when the face mask is in use, wherein the body portion comprises an upper section configured to be positioned around the nasal passages of the user when the face mask is in use and a lower section configured to be positioned proximate to a chin of the user when the face mask is in use, and wherein the upper section of the body portion is shaped to conform to the user's nasal passages in order to direct exhaled air from the user's nasal passages downward toward the lower section. In some embodiments, the face mask is configured to allow the exhaled air to exit an interior space defined between the body portion and the user's face near the chin of the user. In some embodiments, the lower section of the body portion is configured to provide a gap between the body portion and the user's face near the chin, said gap allowing the exhaled air to exit the interior space proximate the chin.

For purposes of summarizing the disclosure, certain aspects, advantages, and features of the technology have been described herein. Not necessarily any or all such advantages are achieved in accordance with any particular embodiment of the technology disclosed herein. No aspects of this disclosure are essential or indispensable. Neither the preceding summary nor the following detailed description purports to limit or define the scope of protection. The scope of protection is defined by the claims.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1A illustrates an embodiment of a face mask placed over a portion of the user's face in accordance with aspects of the present disclosure.

FIG. 1B illustrates an embodiment of a face mask including a display in accordance with aspects of the present disclosure.

FIG. 1C illustrates a schematic diagram of certain features which can be included in the face mask of FIG. 1A in accordance with aspects of the present disclosure.

FIG. 2A illustrates a front perspective view of another embodiment of a face mask secured to a user's face in accordance with aspects of the present disclosure.

FIG. 2B illustrates a partially exploded view of that which is shown in FIG. 2A in accordance with aspects of the present disclosure.

FIG. 2C illustrates a back perspective view of the face mask of FIG. 2A in accordance with aspects of the present disclosure.

FIG. 2D illustrates an optional battery pack of a harness of the face mask shown in FIG. 2C in accordance with aspects of the present disclosure.

FIG. 2E illustrates an enlarged front perspective view of the face mask of FIG. 2A with a portion of the face mask shown in dotted lines in accordance with aspects of the present disclosure.

FIG. 3A illustrates a front perspective view of another embodiment of a face mask secured to a user's face in accordance with aspects of the present disclosure.

FIG. 3B illustrates an enlarged view of the face mask of FIG. 3A in accordance with aspects of the present disclosure.

FIG. 3C illustrates a side view of the face mask of FIG. 3A secured to the user's face in accordance with aspects of the present disclosure.

FIG. 3D illustrates a enlarged view of a portion of the face mask shown in FIG. 3C in accordance with aspects of the present disclosure.

FIGS. 3E-3H illustrate various views of a filter of the face mask of FIG. 3A in accordance with aspects of the present disclosure.

FIG. 3I illustrates an alternative embodiment of the face mask of FIG. 3A in accordance with aspects of the present disclosure.

FIGS. 3J-3L illustrate various views of a filter assembly in accordance with aspects of the present disclosure.

FIG. 4A illustrates a front perspective view of another embodiment of a face mask secured to a user's face in accordance with aspects of the present disclosure.

FIG. 4B illustrates a side view of the face mask of FIG. 4A secured to the user's face in accordance with aspects of the present disclosure.

FIG. 4C illustrates an enlarged view of the face mask shown in FIG. 4B in accordance with aspects of the present disclosure.

FIG. 4D illustrates a schematic diagram of certain features of a capnograph module in accordance with aspects of the present disclosure.

FIG. 5 illustrates a front perspective view of another embodiment of a face mask secured to a user's face in accordance with aspects of the present disclosure.

FIG. 6 illustrates a front perspective view of another embodiment of a face mask secured to a user's face in accordance with aspects of the present disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed embodiments and/or uses and obvious modifications and equivalents thereof. Thus, it is intended that the scope of this disclosure should not be limited by any particular embodiments described below. The features of the illustrated embodiments can be modified, combined, removed, and/or substituted as will be apparent to those of ordinary skill in the art upon consideration of the principles disclosed herein.

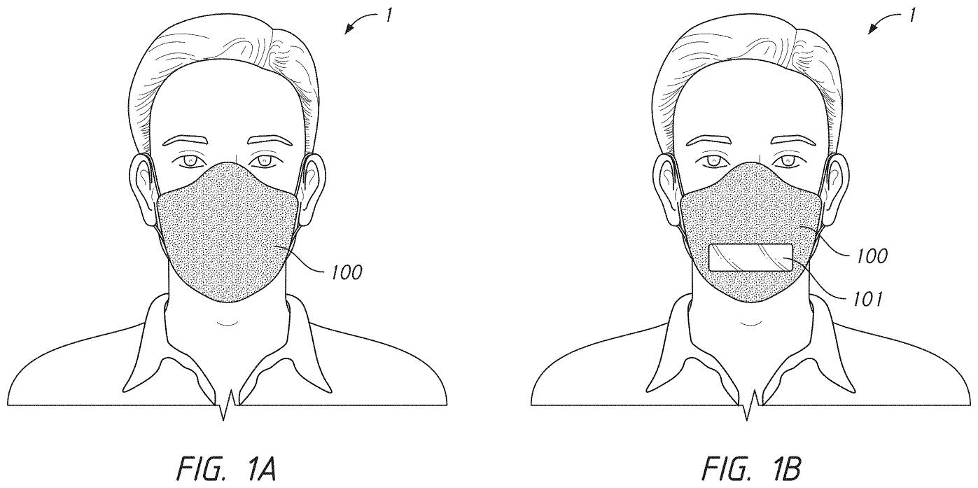

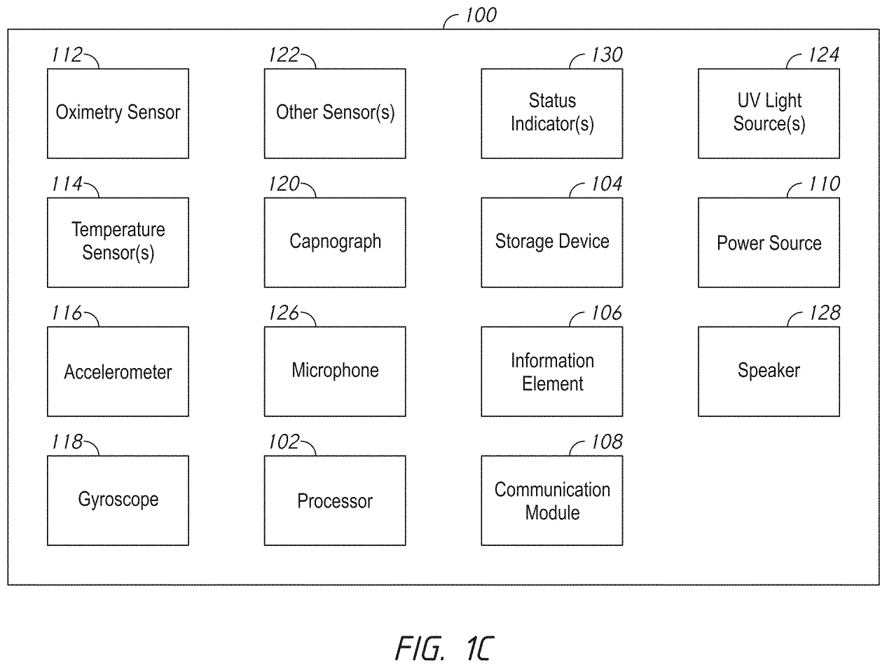

FIG. 1A illustrates a face mask 100 secured to a portion of a face of a user 1. The face mask 100 can include a body portion which can cover a user's mouth and/or one or more nasal passages of the user 1. The face mask 100 can be configured to secure (for example, removably secure) to the user's face. For example, the face mask 100 can include one or more straps which can be connected to the body portion of the face mask 100 and can secure to and/or around ear(s) and/or another portion of a head of the user 1. Alternatively, the face mask 100 (or portions thereof) can secure to the user's face without the use of a strap. For example, the body portion of the face mask 100 can include an adhesive material that allows the face mask 100 to adhere to the user's skin (for example, around the mouth and/or a portion of the nose of the user 1. Such adhesive material can be disposed along a perimeter of the body portion of the face mask 100, for example. Such adhesive material can be a medical grade adhesive, for example. In some implementations, the body portion of the face mask 100 forms a seal around the user's mouth and/or a portion of the user's nose when the face mask 100 is secured to the user's face by the one or more straps and/or by securement (for example, adhesion) of the body portion of the mask 100 to the user's skin.

The face mask 100 (for example, the body portion of the face mask 100) can include an inner surface and an outer surface opposite the inner surface. The inner surface can face toward the user's face and/or skin when the face mask 100 is secured to the user 1 and the outer surface can face away from the user's face and/or skin when the face mask 100 is secured to the user 1. The face mask 100 (or portions thereof) can be made from a variety of materials, such as plastic and/or fabric. The face mask 100 (or portions thereof) can filter and/or block fluid particles (such as fluid particles exiting the user's mouth). The face mask 100 can thus prevent transmission of airborne germs to and/or from the user's mouth.

Advantageously, the face mask 100 can include various electronic components that enable the face mask 100 to carry out processing of various physiological parameters of the user 1 and/or that enable the face mask 100 to interact with various physiological measurement sensors coupled with and/or integrated within the face mask 100 and/or with separate computing devices (such as a mobile phone). FIG. 1C illustrates a schematic diagram of certain features which can be incorporated in face mask 100. The face mask 100 can include any or all of processor 102, storage device 104, information element 106, and/or communication module 106.

The processor 102 can be configured, among other things, to process data (for example, data received from one or more physiological sensors integral and/or coupled with the face mask 100), execute instructions to perform one or more functions, and/or control the operation of the face mask 100. For example, the processor 102 can process physiological data obtained from one or more physiological sensors of the face mask 100 and can execute instructions to perform functions related to storing and/or transmitting such physiological data. As another example, the processor 102 can process data received from one or more physiological sensors of the face mask 100, such as any or all of oximetry sensor 112, temperature sensor(s) 114, accelerometer 116, gyroscope 118, capnograph 120, and/or any other sensor(s) 122 of the face mask 100. Each of oximetry sensor 112, temperature sensor(s) 114, accelerometer 116, gyroscope 118, capnograph 120, and other sensor(s) 122 are discussed in more detail below. The processor 102 can execute instructions to perform functions related to storing and/or transmitting any or all of such received data.

In some embodiments, the face mask 100 includes a circuit board which includes the processor 102, among other things. The circuit board can be mounted to and/or within the body portion of the face mask 100, for example. The face mask 100 can include an electronic module or other type of structure that is coupled to the body portion of the face mask 100 and which can contain various electronic components of the face mask 100 such as those discussed above. Such electronic module or other type of structure can be positioned exterior to the body portion of the face mask 100 and/or interior to the body portion of the face mask 100 (for example, can be positioned between the face mask 100 and the user's face when the face mask 100 is secured to the user 1.

The storage device 104 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data or other types of data (for example, motion and/or location data) obtained from the face mask 100, for example.

In some implementations, the face mask 100 includes an information element 106. The information element 106 can be a memory storage element that stores, in non-volatile memory, information used to help maintain a standard of quality associated with the face mask 100. Illustratively, the information element 106 can store information regarding whether the face mask 100 has been previously activated and whether the face mask 100 has been previously operational for a prolonged period of time, such as, for example, one, two, three, four, five, six, seven, or eight or more hours. The information stored in the information element 106 can be used to help detect improper use and/or re-use of the face mask 100, for example.

The communication module 108 can facilitate communicate (via wired and/or wireless connection) between the face mask 100 (and/or components thereof) and separate devices, such as separate monitoring and/or mobile devices. For example, the communication module 108 can be configured to allow the face mask 100 to wirelessly communicate with other devices, systems, and/or networks over any of a variety of communication protocols. The communication module 108 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 108 can allow data and/or instructions to be transmitted and/or received to and/or from the face mask 100 and separate computing devices. The communication module 108 can be configured to transmit (for example, wirelessly) processed and/or unprocessed physiological or other information to separate computing devices, which can include, among others, a mobile device (for example, an iOS or Android enabled smartphone, tablet, laptop), a desktop computer, a server or other computing or processing device for display and/or further processing, among other things. Such separate computing devices can be configured to store and/or further process the received physiological and/or other information, to display information indicative of or derived from the received information, and/or to transmit information—including displays, alarms, alerts, and notifications—to various other types of computing devices and/or systems that may be associated with a hospital, a caregiver (for example, a primary care provider), and/or a user (for example, an employer, a school, friends, family) that have permission to access the subject's data. As another example, the communication module 108 of the face mask 100 can be configured to wirelessly transmit processed and/or unprocessed obtained physiological information and/or other information (for example, motion and/or location data) to a mobile phone which can include one or more hardware processors configured to execute an application that generates a graphical user interface displaying information representative of the processed or unprocessed physiological and/or other information obtained from the face mask 100. The communication module 108 can be and/or include a wireless transceiver.

With continued reference to FIG. 1C, the face mask 100 can include a power source 110. Such power source 110 can be, for example, a battery. Such battery can be rechargeable or non-rechargeable. The power source 110 can provide power for the hardware and/or electronic components of the face mask 100 described herein. The power source 110 can be, for example, a lithium battery. Additionally or alternatively, the face mask 100 can be configured to obtain power from a power source that is external to the face mask 100. For example, the face mask 100 can include or can be configured to connect to a cable which can itself connect to an external power source to provide power to the face mask 100. Such implementations may be advantageous in certain situations where it is desirable for the face mask to only be employed when the user is in a stationary setting, for example, in a hospital bed. In some implementations, the face mask 100 does not include power source 110.

Advantageously, as discussed above, the face mask 100 can include and/or can be coupled with various physiological sensors (which may also be referred to as “physiological measurement devices”) that can be used to measure one or more physiological parameters of the user 1. For example, in some embodiments, the face mask 100 includes an oximetry sensor 112 (which may also be referred to as a “pulse oximetry sensor” or an “optical sensor”). Oximetry sensor 112 can be integrated into the face mask 100 (such as into the body portion of the face mask 100). Oximetry sensor 112 can include one or more emitters and one or more detectors for obtaining physiological information indicative of one or more blood parameters of the user 1. These parameters can include various blood analytes such as oxygen, carbon monoxide, methemoglobin, total hemoglobin, glucose, proteins, glucose, lipids, a percentage thereof (e.g., concentration or saturation), and the like. Oximetry sensor 112 can also or alternatively be used to obtain a photoplethysmograph, a measure of plethysmograph variability, pulse rate, a measure of blood perfusion, and the like. Information such as oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, can be obtained from the pulse oximetry sensor and data related to such information can be transmitted to a processor of oximetry sensor 112 and/or to processor 102 of the face mask 100 (discussed above) which can be disposed within and/or coupled with the body portion of the face mask 100, for example. Oximetry sensor 112 can be similar or identical to any of the physiological sensors described in U.S. Pat. No. 10,993,662, titled “Nose Sensor,” U.S. Pat. No. 7,341,559, titled “Pulse Oximetry Ear Sensor,” U.S. Pat. No. 10,849,554, titled “Nose Sensor,” U.S. Pat. Application No. 63/187,071, filed on May 11, 2021, titled “Optical Physiological Nose Sensor,” and U.S. Pat. Application No. 63/193,415, filed on May 26, 2021, titled “Optical Physiological Nose Sensor,” all of which are hereby incorporated by reference in their entireties. As another example, oximetry sensor 112 can be a reflectance type oximetry sensor, such as that described in U.S. Pat. No. 10,448,871, titled “Advanced Pulse Oximetry Sensor,” which is hereby incorporated by reference in its entirety. In some embodiments, oximetry sensor 112 includes one or more emitters and one or more detectors arranged in a reflectance arrangement and can be utilized to measure blood perfusion from cheeks or other portions of the face that are in contact with the face mask 100.

The face mask 100 can include and/or can be coupled with other physiological sensors in addition or as an alternative to oximetry sensor 112. For example, the face mask 100 can include one or more temperature sensors 114. In some implementations, the temperature sensor(s) 114 comprises a thermistor. The one or more temperature sensors 114 can be disposed along the inner and/or outer surfaces of the body portion of the face mask 100, for example. The one or more temperature sensors 114 can be positioned along a perimeter of the body portion of the face mask 100. In some cases, the face mask 100 includes a temperature sensor 114 that is positioned adjacent skin of the user's nose when the face mask 100 is secured to the user. The face mask 100 can include a first temperature sensor 114 configured to determine a temperature of skin of the user and a second temperature sensor 114 that is configured to determine an ambient temperature (for example, at a location spaced from the user's skin). In some implementations, temperature values determined using such first and second temperature sensor can be utilized in a manner similar to that described in U.S. application Ser. No. 17/206,907, titled “Wearable Device for Noninvasive Body Temperature Measurement,” filed Mar. 19, 2021, which is hereby incorporated by reference is its entirety. In addition or as an alternative to temperature sensors configured and/or operably positioned by the face mask 100 to measure skin temperature and/or ambient temperature, the face mask 100 can include one or more temperature sensors configured to measure temperature of gases exhaled by the wearer. For example, in some implementations, face mask 100 includes one or more temperature sensors positioned on the face mask 100 in an exit path or paths of exhaled gas of the wearer in order to capture exhaled breath temperature (EBT). In some cases, variation (for example, an increase) of EBT can be indicative of a status of the wearer of the face mask 100, for example, that the wearer has a fever, an infection (for example, viral respiratory infection), asthma, COPD, among other things.

The face mask 100 can include one or more sensors for measuring motion, orientation, and/or location of a user. Any of such motion sensors can be configured to determine motion, orientation, and/or location of a user and/or data from any of such motion sensors can be utilized by processor 102 of face mask 100 to determine motion, orientation, and/or location of the user. For example, the face mask 100 can include a motion sensor that can measure static (for example, gravitational force) and/or dynamic acceleration forces (for example, forces caused by movement or vibration of the motion sensor). By measuring one or both of static and dynamic acceleration forces, such motion sensor can be used to calculate movement or relative position of the face mask 100. Such motion sensor can be an AC-response accelerometer (for example, charge mode piezoelectric accelerometer, voltage mode piezoelectric accelerometer), a DC-response accelerometer (for example, capacitive accelerometer, piezoresistive accelerometer), a microelectromechanical system (MEMS) gyroscope, a hemispherical resonator gyroscope (HRG), vibrating structure gyroscope (VSG), a dynamically tuned gyroscope (DTG), fiber optic gyroscope, and the like. Such motion sensor can measure acceleration forces in one-dimension, two-dimensions, or three-dimensions. With calculated position and movement data, users of the face mask 100 and/or others (for example, care providers) may be able to map the positions or movement vectors of the face mask 100. Any number of motion sensors can be used collect sufficient data to determine position and/or movement of the face mask 100.

For example, with reference to FIG. 1C, the face mask 100 can include an accelerometer 116. The accelerometer 116 can be, for example, a three-dimensional (3D) accelerometer. The accelerometer 116 can be similar or identical to any of those discussed in U.S. Pat. No. 10,226,187, titled “Patient-Worn Wireless Physiological Sensor,” which is incorporated by reference herein in its entirety. With continued reference to FIG. 1C, the face mask 100 can include a gyroscope 118. The gyroscope 118 can be similar or identical to any of those discussed in U.S. Pat. No. 10,226,187. Some implementations of face mask 100 can interact and/or be utilized with any of the physiological sensors and/or systems described in U.S. Pat. No. 10,226,187, for example, to determine whether a user has fallen and/or orientation of a user. In some implementations, face mask 100 can be configured to determine and/or keep track of movement of a user. For example, face mask 100 can be configured to determine and/or keep track of steps and/or distance traveled by a user based on data from the accelerometer 116, gyroscope 118, and/or a magnetometer (for example, a compass).

The face mask 100 can include and/or can be coupled with a capnograph 120 that can be used to measure and/or monitors aspects of the user's respiratory system and health. For example, the face mask 100 can include and/or be configured to connect to any of the respiratory gas measuring devices described in U.S. Pat. No. 10,532,174, titled “Assistive Capnography Device,” which is incorporated by reference herein in its entirety. In some embodiments, the face mask 100 can be similar to the breathing mask which couples to the airway adapter and which forms part of the system described and illustrated in U.S. Pat. No. 10,532,174. In some embodiments, capnograph 120 is similar or identical to the EMMA® Capnograph manufactured and sold by Masimo Corporation. Implementations of face mask 100 including capnograph 120 can enable measurements of physiological parameters such as end tidal respiratory gases including oxygen (O₂), carbon dioxide (CO₂), and nitrous oxide (N₂O), among others, as well as respiratory rate.

With continued reference to FIG. 1C, the face mask 100 can include one or more other sensor(s) 122. Such other sensors 120 can be, for example, a humidity sensor, an impedance sensor, an acoustic/respiration sensor, among others. In some implementations, the face mask 100 can connect to a physiological measurement device that monitors electrical activity of a heart of the user 1. For example, the face mask 100 can wirelessly (via a wireless transceiver of the face mask 100) or via a wired connection (for example, a cable) connect to a physiological measurement device that measures electrocardiogram data (ECG) of the user, which can secure to the user's chest. Such physiological measurement device can be an ECG device such as that described in U.S. Pat. Pub. No. 2020/0329993, titled, “Electrocardiogram Device,” which is hereby incorporated by reference in its entirety.

In some cases when users wear a face mask, volume and/or clarity of the user's voice is impaired and/or altered (for example, muffled). In some implementations, face mask 100 includes a microphone 126 that can be utilized to alleviate such impairment and/or alteration. Microphone 126 can be positioned within various locations of the face mask 100 and can detect sound, for example, of the user's voice. In some implementations, the microphone 126 can convert detected sound to digital signals for analysis and/or processing. In some implementations, microphone 126 can generate and/or transmit one or more signals responsive to detected sound to processor 102. In some implementations, face mask 100 includes a speaker 128. In some implementations, processor 102 can be in communication with both of microphone 126 and speaker 128 and can instruct speaker 128 to output sounds based on that which is detected by the microphone 126. In some implementations, face mask 100 is configured to wirelessly communicate with a separate device, such as a mobile phone and/or an auricular device, which can facilitate audio transmission and/or output. Such auricular device can be similar or identical to auricular device 190 described below. In some implementations, audio communication between two users wearing face mask 100 can be facilitated using microphone 126 and speakers 128 in the face mask 100 and/or in separate devices in communication with the face masks 100. By way of non-limiting example, a first user's voice can be detected by a first microphone 126 in a first face mask 100 worn by the first user and detected sounds can be outputted by a first speaker 128 of the first face mask 100 and/or wirelessly transmitted to a second face mask 100 of a second user for outputting by an auricular device coupled to the second face mask 100. Various other techniques can be utilized using such configurations to facilitate communication between two users wearing face masks 100.

In some implementations, the face mask 100 includes UV light source(s) 124 that can illuminate and/or disinfect portions of the face mask 100. For example, the UV light source(s) 124 can illuminate and/or disinfect portions of an interior space defined by the face mask 100 when secured to the wearer's face and/or portions of the wearer's skin in and/or around the mouth and nasal passages. UV light source(s) 124 can be operated continuously or periodically/intermittently. In some cases, UV light source(s) 124 extend along a perimeter of the face mask 100 (or a portion of such perimeter), for example, along an edge of the face mask 100 at or near where the face mask 100 contacts the wearer's face.

In some implementations, the face mask 100 includes status indicator(s) 130. Status indicator(s) 130 can be a light source, such as light-emitting diode (LED). Status indicator(s) 130 can visually indicate a status of the face mask 100, for example, that the face mask 100 is operational (is in an “on” mode), a charging status of the face mask 100 (for example, where the face mask 100 is configured to receive power from an external power source via a cable or wireless inductive charging), a battery life, among other things. For example, status indicator 130 can illuminate a first color (for example, green) when the face mask is “on” and/or when a life of power source 110 is above a threshold value or percentage. As another example, status indicator 130 can illuminate a second color (for example, red) when the face mask 100 is “off” and/or when a life of power source 110 is too low (for example, below such threshold). Processor 102 can be in communication with power source 110 and/or other components of the face mask 100 and can instruct and/or otherwise cause status indicator 130 to operate in such manner. In addition or as an alternative to the above, status indicator(s) 130 can be utilized to visually indicate a status of the wearer of the face mask 100. As discussed above, face mask 100 can include and/or be coupled with one or more physiological sensors that can be used for determining one or more physiological parameters of the wearer. In some implementations, processor 102 is configured to instruct and/or otherwise cause status indicator 130 to illuminate a certain color, change color, and/or operate otherwise (for example, blink) responsive to determinations of one or more physiological parameters of the wearer and/or responsive to determination that a status of the wearer (for example, based on such determined parameters) is not optimal. Such status of the wearer can be, for example, temperature above a threshold that is indicative of a fever, oxygen saturation below a threshold indicative of hypoxemia (for example, among other things), abnormally fast or slow heart rate indicative of tachycardia or bradycardia (respectively), abnormal levels of end-tidal carbon dioxide (EtCO₂) indicative of respiratory problems, among other things.

In some implementations, the face mask 100 can determine and/or display an emotion of a user wearing the face mask 100. This can be advantageous where the face mask 100 covers a portion of the user's face and therefore impairs a third party's ability to visually determine the user's facial expressions (which can indicate the user's emotion). The face mask 100 can include one or more cameras that can capture one or more images of a portion (or portions) of the user's face. The one or more cameras can be coupled to the body portion of the face mask 100. For example, the one or more cameras can be connected to a side or surface of the body portion of the face mask 100 that faces toward the user's face when the face mask 100 is in use. The one or more cameras can capture one or more images of portions of the user's face at or near the user's mouth, for example. The face mask 100 can include a display 101 that can be coupled to the body portion of the face mask 100 (see FIG. 1B). For example, the display 101 can be connected to a side or surface of the body portion that faces away from the user's face when the face mask 100 is in use. Processor 102 can be operatively connected to the one or more cameras and the display 101. Processor 102 can receive the captured one or more images from the one or more cameras and can display the one or more images on the display 101. This can advantageously allow a third party interacting and/or otherwise within a certain proximity of the user wearing the face mask 100 to see facial expressions of the user as if the face mask 100 was not being worn.

In some implementations, the face mask 100 includes a plurality of cameras. For example, the face mask 100 can include two, three, four, five, or six or more cameras configured to capture one or more images of portions of the user's face. As another example, the face mask 100 can include a first camera configured to capture one or more images of a portion of the user's face proximate a right corner of the user's mouth, a second camera configured to capture one or more images of a portion of the user's face proximate to a left corner of the user's mouth, and a third camera configured to capture one or more images of the user's mouth. In some cases, narrowing the field of view for each of these three cameras in such manner can allow the processor 102 to more easily compare captured images with reference images in a storage device of the face mask 100 in order to determine corresponding facial expressions of the user, as discussed further below.

In some implementations, display 101 is a flexible display. The flexible display can be an electronic paper display, an organic liquid crystal display (LCD), and/or an organic light-emitting diode (LED) display. Such flexible display can be coupled with the body portion of the face mask 100, for example, on a side or surface of the body portion that faces away from the user when the face mask 100 is worn so as to be viewable by third parties interacting and/or otherwise in the vicinity of the wearer of the face mask 100. In some implementations, the face mask 100 can determine an emotion of the user wearing the face mask 100 and can display a visual representation of the emotion on display 101. The face mask 100 can include one or more cameras which can be operatively connected to processor 102 and processor 102 can receive the captured one or more images from the one or more cameras and determine a facial expression of the user based on the received, captured one or more images. In some implementations, processor 102 can generate a visual representation of the facial expression on display 101 based on the determined facial expression.

In some implementations, processor 102 can compare the one or more images received from the one or more cameras to one or more reference images stored in storage device 104. Storage device 104 can include reference images associated with various facial expressions of the user and/or a sample population of users (which can be indicative of an emotion of the user). For example, storage device 104 can include reference images associated with facial expressions such as smiling, laughing, crying, anger, confusion, frustration, among others. In some implementations, processor 102 compares the one or more images received from the one or more cameras to one or more reference images stored in storage device 104 and determines, based on one or more comparisons or groups or comparisons, whether the one or more images correspond with a particular facial expression. In some implementations, the one or more cameras continuously or periodically capture and send images of a portion (or portions) of the user's face to processor 102, and, in response, processor 102 continuously or periodically determines, based on the received images, facial expressions of the user. Additionally, processor 102 can continuously or periodically generate a visual representation of the determined facial expressions on display 101. Such visual representation can be, a representation of a smiling mouth (for example, where the facial expression is determined to be a smile). Such visual representation can be a cartoon or other graphic representing a smiling, laughing, or angry facial expression, for example.

The face mask 100 can include one or more sensors responsive to a motion of a portion (or portions) of the user's face. Such one or more sensors can be and/or include accelerometer 116 and/or gyroscope 118 discussed above. The one or more sensors can be coupled with the body portion of the face mask 100. For example, the one or more sensors can be connected the body portion proximate an edge of the body portion which can be positioned near skin of the user's face when the face mask 100 is in use. The one or more sensors can generate one or more signals responsive to motion of the portion (or portions) of the user's face. In some implementations, processor 102 receives the one or more signals from the one or more sensors and determines the facial expression of the user based on said received one or more signals. In some implementations, processor 102 determines the facial expression of the user based on one or more signals received from the one or more sensors and also based on one or more images received from the one or more cameras discussed above.

In some implementations, the face mask 100 includes structure to direct exhaled air downward and/or in a direction away from the user's eyes and/or upper face. Such configuration can advantageously minimize or prevent exhaled air from drying out the user's eyes and/or from fogging up glasses worn by the user. For example, in some implementations the body portion of the face mask 100 is shaped to conform to the user's nose such that exhaled air from the user's nostrils is directed downward toward the user's chin and/or otherwise away from the nose and upper portion of the user's face. In some implementations, an upper section of the body portion of the face mask 100 is shaped to conform to the user's face at and/or around the user's nose and a lower section of the body portion of the face mask 100 is shaped to direct exhaled air toward a bottom of the face mask 100 and out of the interior space defined between the user's face and the face mask 100. In some implementations, the lower section of the body portion of the face mask 100 is shaped to include a gap or spacing between the user's face and the face mask 100 in order to provide an exit pathway for exhaled air, for example, at or near the user's chin.

As another example, in some implementations, the face mask 100 can include a cannula with one or more prongs sized and/or shaped to fit within the user's nostrils and structured to direct exhaled air downward (e.g., toward the user's chin) and/or outward from the interior space defined between the user's face and the face mask 100. Such structure can, similar to that described above, advantageously minimize or prevent exhaled air from exiting out an upper section of the face mask 100 above or around the user's nose and/or toward the user's eyes. In some implementations, the cannula is coupled to the body portion of the face mask 100, for example, connected to a side or surface of the body portion that faces toward the user's face when the face mask 100 is in use. In some implementations, the cannula includes two prongs, each respective prong sized and/or shaped to be received and/or secure within a different one of the user's nostrils. The cannula and/or the one or more prongs of the cannula can be connected to the body portion of the face mask 100 and oriented to direct exhaled air from the user's nostrils outward from the interior space defined between the face mask 100 and the user's face and/or towards a portion of the interior space (for example, towards a bottom portion of the interior space near a bottom end of the face mask 100 positioned proximate the user's chin when the face mask 100 is in use). In some implementations, the one or more prongs are configured to extend through a portion of the face mask 100 to direct exhaled air out of such interior space. For example, the one or more prongs can be coupled to the body portion of the face mask 100 and can extend from a first end configured to be received within the user's nostrils to a second end which is positioned outside such interior space. As another example, the one or more prongs can extend from within the user's nostrils (when the face mask 100 is in use) downward toward a bottom portion of the face mask 100 positioned proximate the user's chin. In some implementations, the prongs can be elongated and can extend along and/or be secured to a side of surface of the body portion of the face mask 100 that faces toward the user's skin, and can further extend toward a bottom portion of the face mask 100 at or near the user's chin when the face mask 100 is in use. Various other configurations of the face mask 100 in addition to those discussed above are possible which advantageously direct exhaled air downward and/or in a direction away from the user's eyes and/or upper face. The cannula and/or one or more prongs discussed above can be similar or identical to those described in U.S. Pat. No. 10,441,196, titled “Nasal/Oral Cannula System and Manufacturing,” which is hereby incorporated by reference in its entirety.

Exhaled breathing gases from the mouth and/or nasal passages are typically saturated with moisture at body temperature. Such moisture can build up in and around a user's face when wearing the face mask 100. Advantageously, in some implementations, the face mask 100 can be made, in part or in whole, of a moisture wicking material and/or fabric which can pull moisture from interior space defined between the face mask 100 and the user's face toward an exterior surface of the face mask 100 where it can efficiently evaporate. For example, the body portion of the face mask 100 can be made (in whole or in part) of a moisture wicking material and/or fabric. Such moisture wicking material can be any of those described in U.S. Pat. No. 9,861,298, titled “Gas Sampling Line,” which is hereby incorporated by reference in its entirety. For example, the moisture wicking material can be or include a hydrophilic member that can transport moisture within the interior space of the face mask 100 to an exterior portion of the face mask 100 (for example, an exterior surface of the body portion of the face mask 100 that faces away from the user during use).

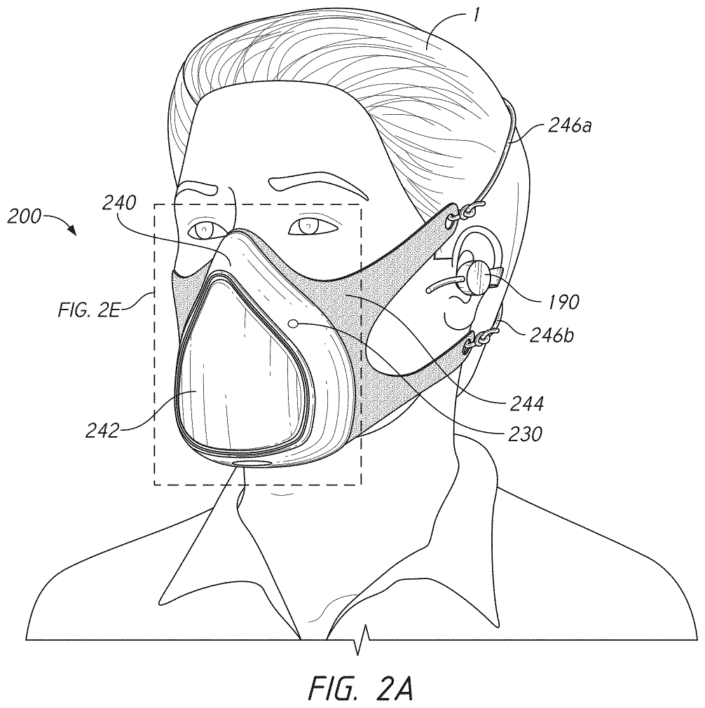

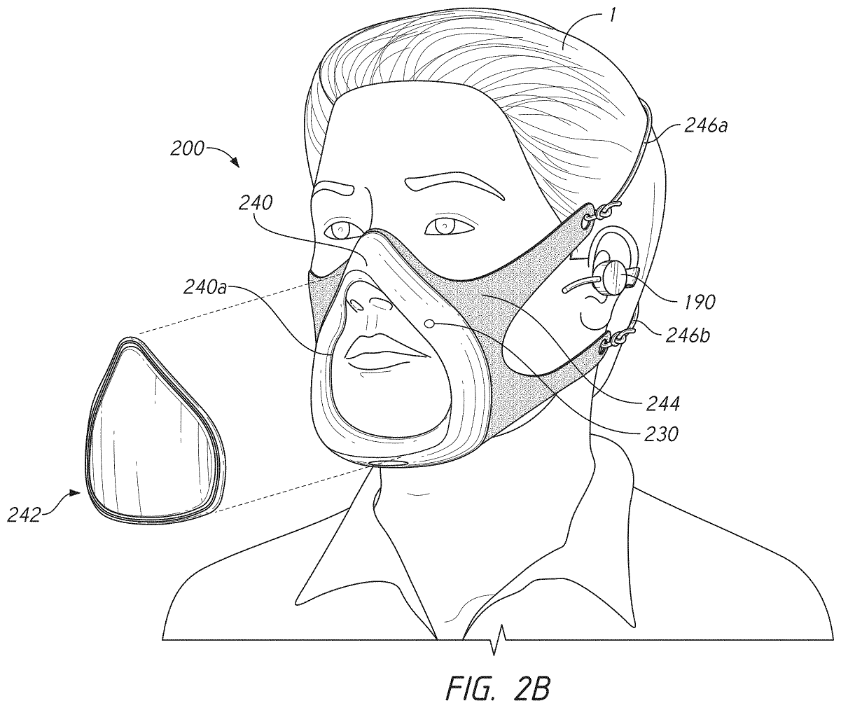

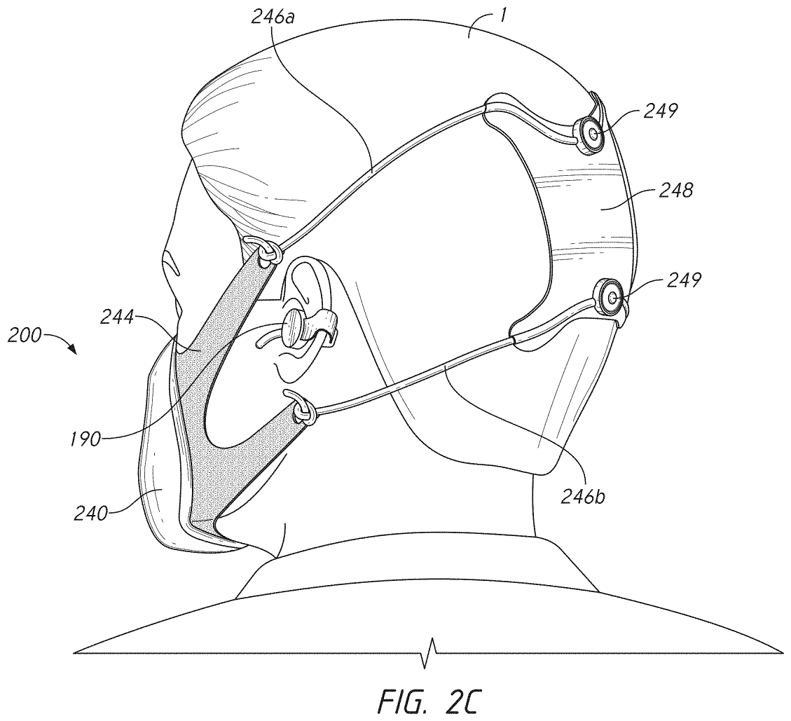

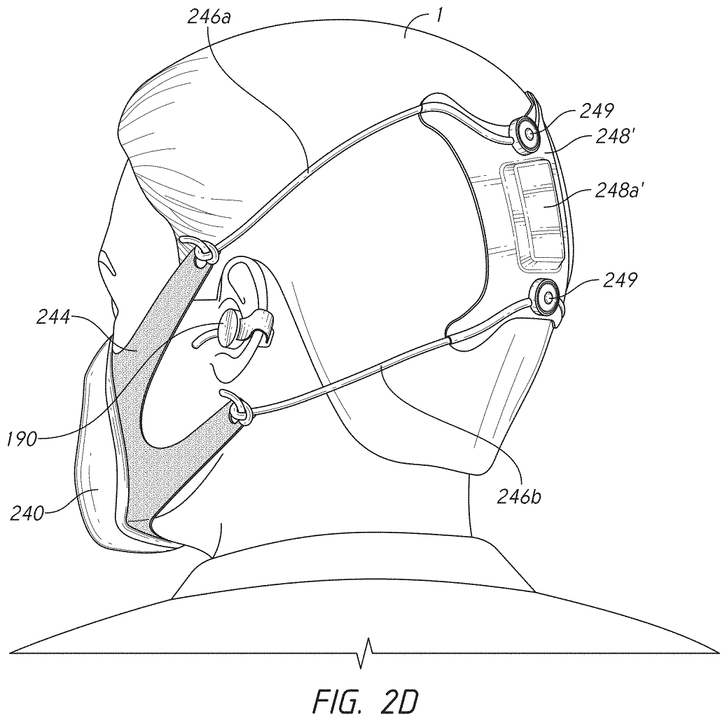

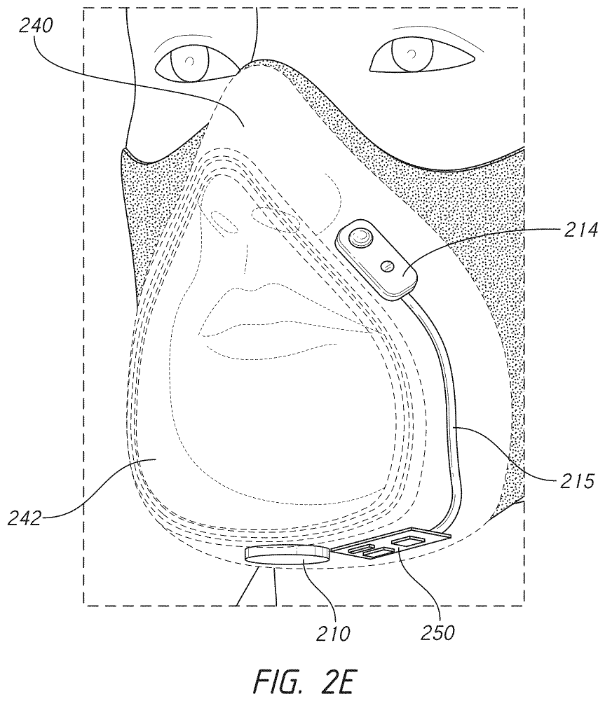

FIGS. 2A-2E illustrate a face mask 200 that can be similar to face mask 100 in some or many respects. Any features or aspects discussed above with respect to face mask 100 can be included and/or incorporated into face mask 200. FIG. 2A illustrates face mask 200 secured to a face of user 1. In some implementations, face mask 200 is configured to cover a mouth and nasal passages of the user 1 when in use. Face mask 200 can include a body portion 240. Body portion 240 can be and/or comprise a frame or other structure that can be rigid and/or flexible and can be sized and/or shaped to surround the mouth and nasal passages of user 1. Body portion 240 can include an upper section configured to be positioned around at least a portion of a nose of the user 1 and/or conform to at least a portion of a shape of the user's nose when the body portion 240 is secured to the user's face. Body portion 240 can include a lower section configured to be positioned at or near a lower portion of the user's face (such as the chin) when the mask 200 is in use.

Body portion 240 can comprise silicone, plastic, and/or rubber, among other materials. Body portion 240 can include and/or be removably coupled with a filter 242 (which can also be referred to as a “screen”) as illustrated in FIG. 2B. In some implementations, filter 242 can mechanically connect to body portion 240 around an opening 240a (see FIG. 2B). Such mechanical connection can be a snap fit or a press fit, for example. Such mechanical connection can allow the filter 242 to be removed and/or replaced. Filter 242 can be an anti-microbial and/or anti-bacterial filter. Filter 242 can be configured to filter out particles from the air (e.g., dusts, mists, fumes, etc.) prior to inhalation by the user and/or can filter out particles in the user's exhaled breath before allowing such exhaled breath to exit an interior space defined by mask 200 when in use. Filter 242 can be a disposable or reusable N95, N100, and/or HEPA type filter. In some implementations, filter 242 can filter out particulates having a size of 0.3 microns or larger. In some implementations, filter 242 is rigid. In some implementations, filter 242 is transparent or semi-transparent (for example, opaque) such that the user's mouth is visible when the filter 242 is in place and the mask 200 is in use. In some implementations, filter 242 is nontransparent. Mask 200 (or portions thereof such as body portion 240 and/or filter 242) can define an interior space when secured to the user's face. Mask 200 can include an inlet configured to allow air to flow into said interior space during inhalation by the user and can include an outlet configured to allow exhaled gases from the user to flow outside said interior space. Such inlet and/or outlet can occupy the same space. Such inlet and/or outlet can be formed and/or defined by filter 242.

Face mask 200 can include one or more straps 246a, 246b to secure the mask 200 to the user. Such straps 246a, 246b can be coupled with the body portion 240. In some implementations, mask 200 includes a front harness 244 (which may also be referred to as a “harness portion” or “front harness”) connected to and/or around body portion 240 (or a portion thereof) which is positioned between and connects the body portion 240 and the straps 246a, 246b. Harness 244 can extend around all or a portion of body portion 240 and have two pairs of arms extending outward from right and left sides of the body portion 240 and spaced from one another as shown. Each of such pair of extending arms can extend above and below ears of the user 1 when mask 200 is in use as shown. In some implementations such as that shown, each of such pair of extending arms are separated by a U-shaped opening. Ends of such pair of arms can connect to ends of straps 246a, 246b. Harness 244 can be transparent, semi-transparent, or nontransparent. Harness 244 can comprise a fabric (such as a mesh fabric) that is breathable, soft, and/or washable. For example, harness 244 can comprise two ply mesh material having a soft cotton flannel inside. Harness 244 can be stretchable. In some implementations, harness 244 comprises a moisture wicking material configured to wick away moisture and/or allow for quick drying. Harness 244 can comprise an anti-microbial mesh fabric.

With reference to FIG. 2C, in some implementations, mask 200 includes a harness 248 (which may be referred to as a “back harness” or “harness portion”) that can be coupled with straps 246a, 246b and can comfortably secure the mask 200 to the back of the user's head. Harness 248 can distribute the force applied by the straps 246a, 246b on a greater surface area than if the straps 246a, 246b were used alone, which can reduce pressure and increase comfort for the user 1. Harness 248 can include a rectangular shape with four arms extending outward from corners of the rectangular shape each connected to portions of straps 246a, 246b as shown. In some implementations, harness 248 can comprise a flexible and/or stretchable material that allows the harness 248 to conform to a shape of a portion of a back of the user's head (see FIG. 2C). Harness 248 can have a thickness that is less than about 1 inch, less than about 0.9 inch, less than about 0.8 inch, less than about 0.7 inch, less than about 0.5 inch, less than about 0.4 inch, less than about 0.3 inch, less than about 0.2 inch, or less than about 0.1 inch. Such configurations can allow the harness 248 to have a minimal profile thereby minimizing obtrusiveness and increasing user comfort. In some implementations, harness 248 includes strap tightening mechanisms 249 that can allow tightness of straps 246a, 246b coupled to harness 248 to be adjusted, for example, via rotation. Such strap tightening mechanisms 249 can be similar to those sold by Boa Technology Inc., for example. In some implementations, harness 248 is configured to be positioned adjacent hair of the user 1 when mask 200 is secured to user 1, for example, as shown in FIG. 2D. In some implementations, harness 248 is configured to be secured to a portion of the user's head other than adjacent the back of the neck of the user 1 when mask 200 is secured to user 1. Such configurations can provide greater levels of securement which may inhibit the mask 200 from sliding off and/or downward.

FIG. 2D illustrates an embodiment of harness 248′ that includes a battery pack or carrier 248a′. Harness 248′ can be identical to harness 248 except with respect to such battery pack or carrier 248a′. Such battery pack or carrier 248a′ can advantageously be utilized to hold a battery that can be used to replace a battery within mask 200, such as battery 210 discussed further below. In some implementations, mask 200 can be configured to derive power from a battery within such optional battery pack or carrier 248a′ on harness 248′ and a cable can extend from such battery pack or carrier 248a′ to another portion of the mask 200 (for example, body portion 240) to connect and provide power to other components of the mask 200. Such cable can be integral with strap 246a and/or 246b in some implementations, for example. In some implementations, a combined thickness of the harness 248′ and battery pack 248a′ can be less than about 3 inch, less than about 2.9 inch, less than about 2.8 inch, less than about 2.7 inch, less than about 2.6 inch, less than about 2.5 inch, less than about 2.4 inch, less than about 2.3 inch, less than about 2.2 inch, less than about 2.1 inch, less than about 2 inch, less than about 1.9 inch, less than about 1.8 inch, less than about 1.7 inch, less than about 1.6 inch, less than about 1.5 inch, less than about 1.4 inch, less than about 1.3 inch, less than about 1.2 inch, less than about 1.1 inch, less than about 1 inch, less than about 0.9 inch, less than about 0.8 inch, less than about 0.7 inch, or less than about 0.5 inch.

FIG. 2E illustrates an enlarged view of that which is shown in FIG. 2A but with certain portions of the mask 200 (and user's face) shown in dotted lines so as to better illustrate optional internal electronic components that can be integrated into mask 200. Mask 200 can include a battery 210 (for example, a coin cell battery), a circuit board 250, and a cable 215 that can connect circuit board 250 to one or more physiological or other sensors of mask 200. Mask 200 can include a processor, communication module, storage device, and/or information element that can be coupled and/or in communication with circuit board 250, and such processor, communication module, storage device, and/or information element can be similar or identical to processor 102, communication module 108, storage device 104, and/or information element 106 discussed above with reference to mask 100. Mask 200 can include a sensor 214 that can be connected to circuit board 250 via cable 215. Sensor 214 can be integrated into a portion of the mask 200, such as into the body portion 240. In some implementations, sensor 214 is operably positioned within and/or by the body portion 240 such that the sensor 214 is placed adjacent skin on the nose or cheek of the user 1 when mask 200 is in use. Sensor 214 can be and/or comprise an oximetry sensor (for example, a reflective oximetry sensor), temperature sensor(s) (for example, skin and/or breath temperature sensor(s)), and such oximetry sensor and/or temperature sensor(s) can be similar or identical to oximetry sensor 112 and/or temperature sensor(s) 114 discussed above. For example, sensor 114 can comprise an oximetry sensor that includes one or more emitters and one or more detectors arranged in a reflective arrangement over skin of the user's cheek, for example, above and/or around the mouth or lip. Additionally or alternatively, sensor 114 can be comprise a temperature sensor that can be utilize to measure skin temperature at skin of the user's cheek, for example, above and/or around the mouth or lip.

With reference to FIGS. 2A-2B, mask 200 can include a status indicator 230. Status indicator 230 can be positioned on a portion of body portion 240 and can be configured to indicate a status of the mask 200 and/or of the user 1 (for example, based on one or more physiological parameters determined from one or more sensors of mask 200). Status indicator 230 can be similar or identical to status indicator 130 discussed above. Status indicator 230 can be in communication with (for example, connected via a wire) circuit board 250.

Similar to as discussed with respect to mask 100, mask 200 can be configured to communicate with separate devices, for example, via a communication module of mask 200 that can be similar or identical to communication module 108. For example, mask 200 can be configured to communicate with one or more auricular devices 190 that can secure to the user's ear(s). In some implementations, mask 200 can be utilized in a system including auricular device(s) 190. Either or both of mask 200 and auricular device 190 can include one or more physiological sensors usable to measure one or more physiological parameters of the user 1. In some implementations, mask 200 includes a microphone that can be similar or identical to microphone 126. In such implementations, such microphone can be used to transmit sounds from the user 1 to the user's own ear via auricular device(s) 190 and/or to an auricular device 190 and/or other device of another user to facilitate audio communication therebetween. Auricular device 190 can be similar or identical to any of the auricular devices described in U.S. Pat. Application No. 63/222,284, filed Jul. 15, 2021, and titled “Auricular Device,” which is hereby incorporated by reference in its entirety.

Face mask 200 can be configured to communicate with separate devices via a communication module of mask 200 that can be similar or identical to communication module 108. For example, mask 200 can be configured to wirelessly transmit processed and/or unprocessed obtained physiological information to a mobile phone which can include one or more processors configured to execute an application that generates a graphical user interface displaying information representative of the processed or unprocessed physiological and/or other information obtained from mask 200. This can advantageously allow a user 1 to monitor physiological information obtained from mask 200 during use via a separate device (for example, mobile phone). Such physiological information can include, for example, any of that which is described herein.

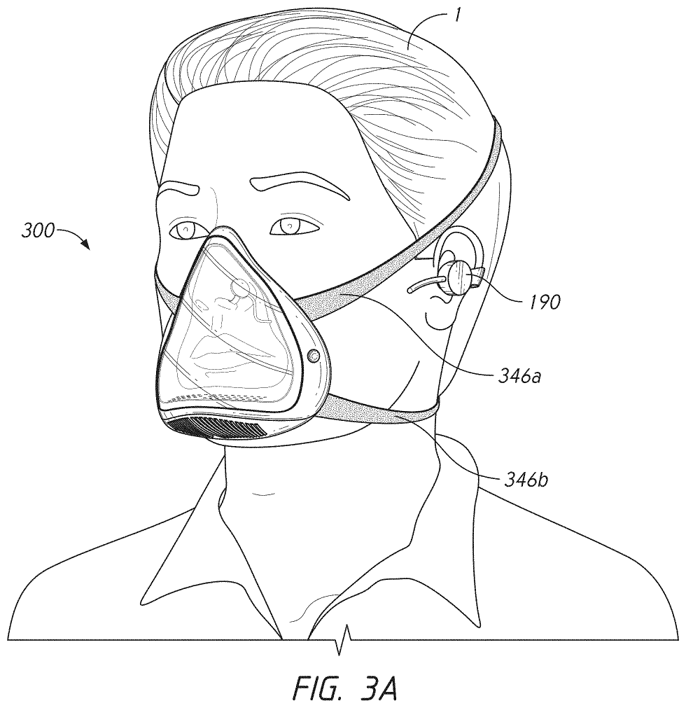

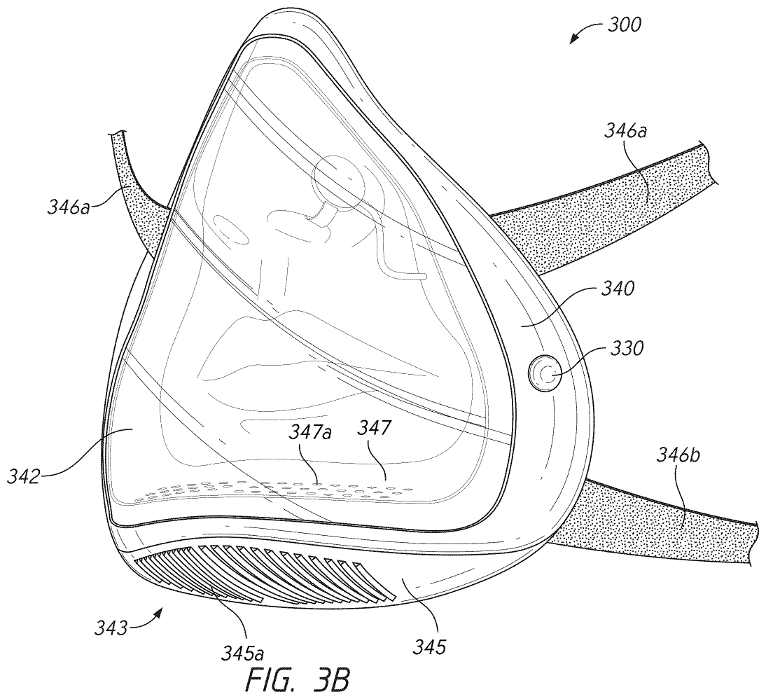

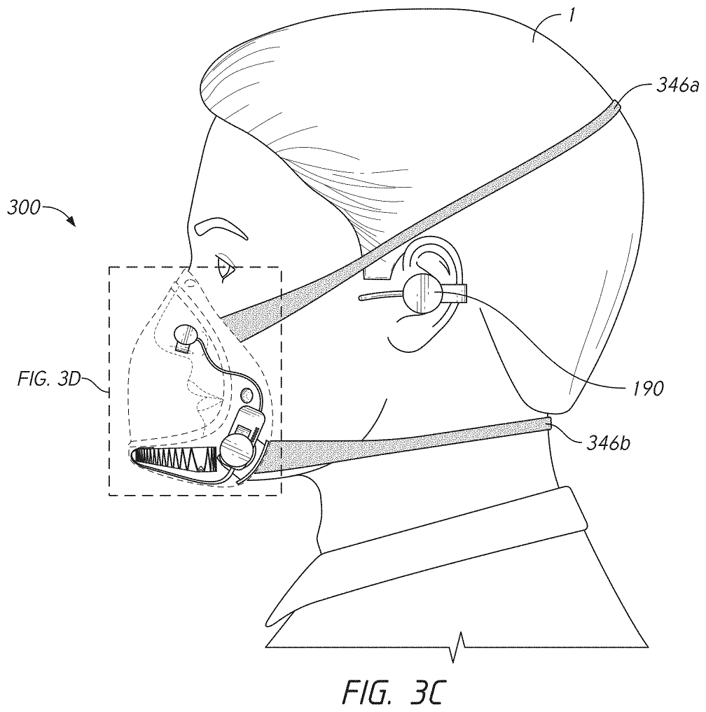

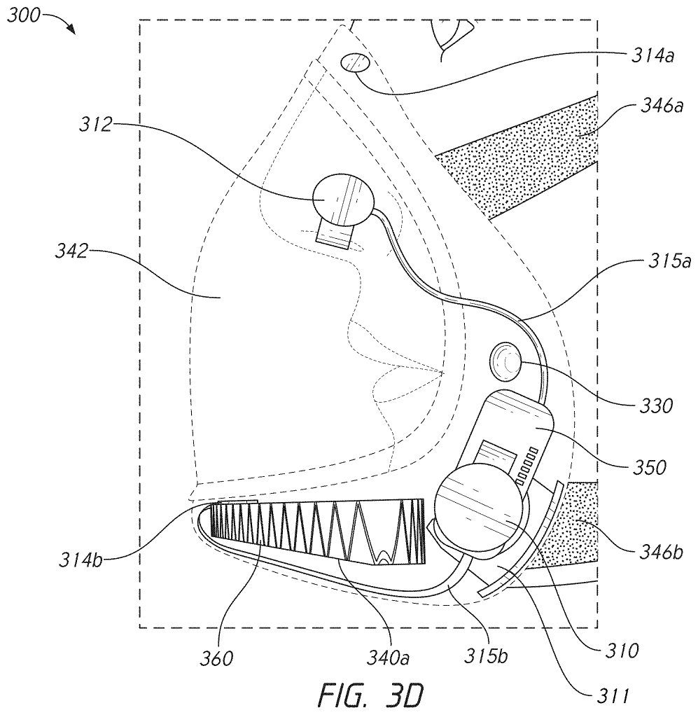

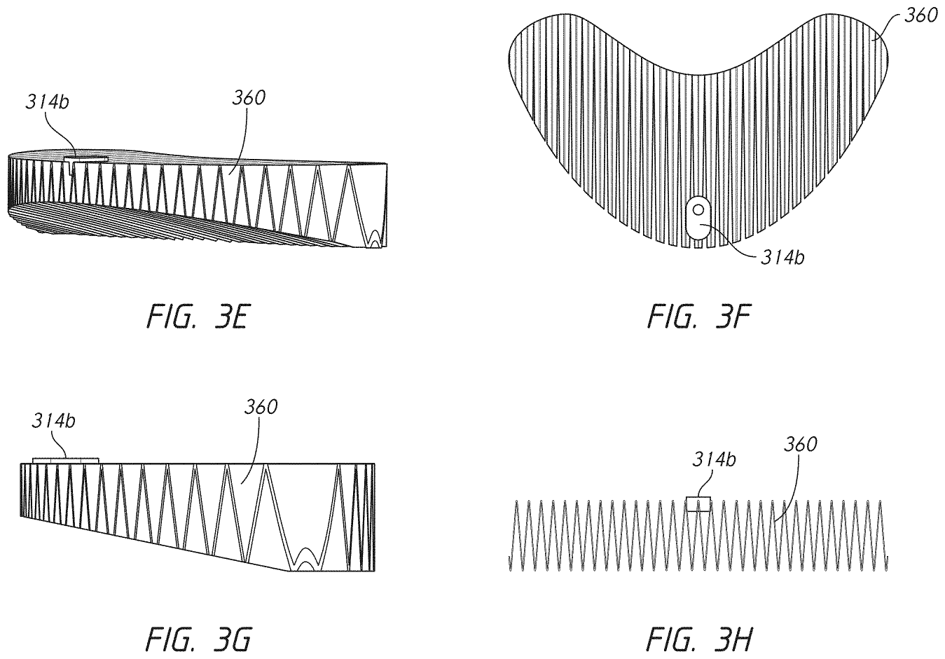

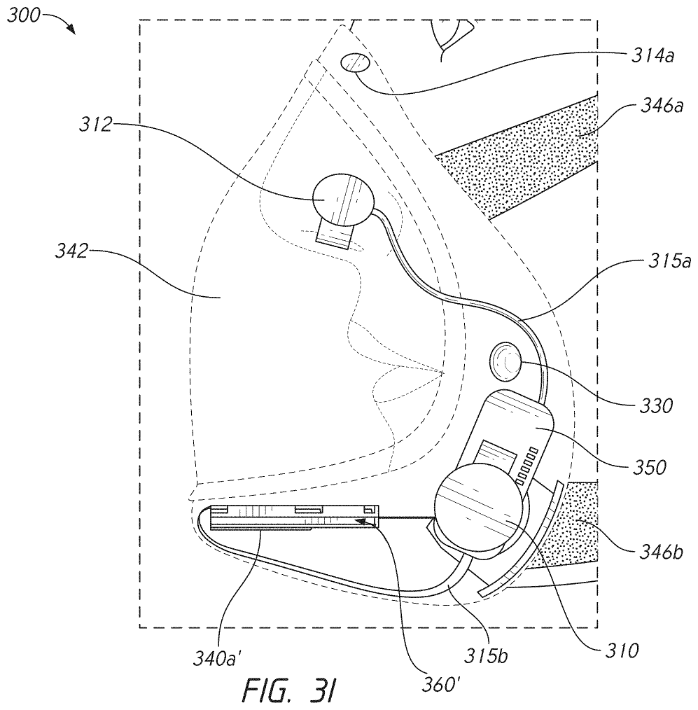

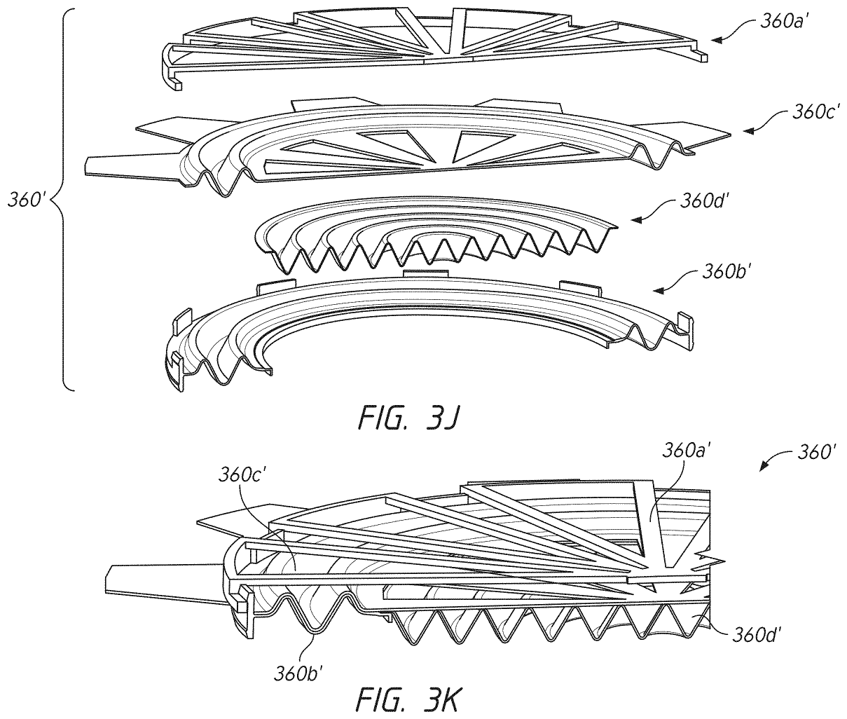

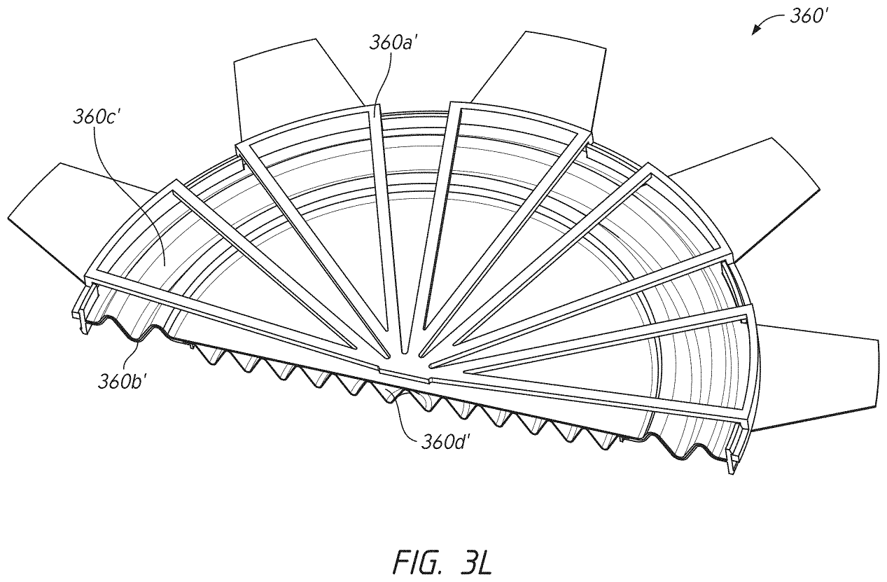

FIGS. 3A-3D illustrate a face mask 300 that can be similar to face mask 100 in some or many respects. Any features or aspects discussed above with respect to face mask 100 can be included and/or incorporated into face mask 300. FIG. 3A illustrates face mask 300 secured to a face of user and FIG. 3B illustrates an enlarged view of face mask 300 separate from the user 1. In some implementations, face mask 300 is configured to cover a mouth and nasal passages of the user 1 when in use. Face mask 300 can include a body portion 340. Body portion 340 can be and/or comprise a frame or other structure that can be rigid and/or flexible and can be sized and/or shaped to surround the mouth and nasal passages of user 1. Body portion 340 can include an upper section configured to be positioned around at least a portion of a nose of the user 1 and/or conform to at least a portion of a shape of the user's nose when the body portion 340 is secured to the user's face. Body portion 340 can include a lower section configured to be positioned at or near a lower portion of the user's face (such as the chin) when the mask 300 is in use. Body portion 340 can include a frame and a seal (which may also be referred to as a “seal member”), where the frame is more rigid than the seal and where the seal forms a seal around a portion of the user's face and/or can define (alone or in combination with other portions of the mask 300) an interior space defined by the mask 300 when in use. Mask 300 can include straps 346a, 346b to secure the mask 300 to the user 1. Such straps 346a, 346b can be coupled with the body portion 340.

Body portion 340 can comprise silicone, plastic, and/or rubber, among other materials. Body portion 340 can include and/or be removably coupled with a window 342 which can be positioned on a front portion of the mask 300 when in use. Window 342 can be transparent or semi-transparent (for example, opaque). Window 342 can comprise plastic or another type of material. Window 342 can be rigid and/or flexible. In some implementations, window 342 comprises an anti-fog, an anti-bacterial, and/or an anti-bacterial coating and/or material. In some implementations, window 342 is hydrophobic. In some implementations, window 342 can mechanically connect to body portion 340 around an opening in body portion 340. Such mechanical connection can be a snap fit or a press fit, for example. Such mechanical connection can allow the window 342 to be removed and/or replaced. Alternatively, in some implementations, window 342 is not removable from body portion 340 (for example, is permanently secured to body portion 340).

Mask 300 (or portions thereof such as body portion 340 and/or window 342) can define an interior space when secured to the user's face. Mask 300 can include an inlet configured to allow air to flow into said interior space (for example, during inhalation by the user) and can include an outlet configured to allow exhaled gases from the user to flow outside said interior space. In some implementations, the inlet and outlet occupy the same space. With reference to at least FIG. 3B, mask 300 can include an inlet/outlet 343. In some implementations, when mask 300 is in use, inlet/outlet 343 faces generally downward, for example, in a direction opposite the mouth and/or nose of the user 1. Such configuration can be advantageous because it directs exhaled breath further away from the window 342 and therefore inhibits potential fogging of window 342. Such configuration can also minimize the potential that exhaled gas is re-breathed by the user 1, for example, when the user 1 is walking or running. Inlet/outlet 343 can be located in a lower section of the body portion 340, for example, that is configured to be positioned at or near a lower portion of the user's face (such as the chin) when the mask 300 is in use. Inlet/outlet 343 can be formed in and/or by the body portion 340 or portions thereof. For example, inlet/outlet 343 can be defined and/or formed by one or more openings in the body portion 340. Such openings can be vents and/or holes in the body portion 340. For example, as shown, inlet/outlet 343 can be defined and/or formed by one or more or a plurality of vents 345a in a bottom wall or surface 345 of the body portion 340 and/or one or more or a plurality of holes 347a in an inner wall or surface 347 of the body portion 340. Bottom wall or surface 345 can be positioned below inner wall or surface 347, for example, when mask 300 is worn by user 1. Bottom wall or surface 345 can face downward and/or toward the ground when mask 300 is worn by user 1. Bottom wall or surface 345 and inner wall or surface 347 can be spaced from one another by a gap, which can define cavity 340a, described further below. In some implementations, vents 345a have a linear shape. In some implementations, holes 347a have a circular shape.

With reference to FIG. 3D which illustrates an enlarged side view of mask 300 with external portions of mask 300 and portions of the user's face shown in dotted lines, body portion 340 can include a cavity 340a that can be positioned and/or formed between such bottom wall or surface 345 and inner wall or surface 347 of body portion 340. Mask 300 can include a filter 360 positioned within cavity 340a and/or between such bottom wall or surface 345 and inner wall or surface 347. FIGS. 3E-3H illustrate perspective, top, side, and back views (respectively) of filter 360. FIGS. 3E-3H also illustrate temperature sensor 314b that can be positioned atop and/or secured to filter as discussed further below.

Body portion 340 (and/or a portion thereof such as a lower section of body portion 340) can be sized and/or shaped to conform to a portion of the user's lower face (for example, chin). In some implementations, with reference to FIG. 3F, filter 360 can have an arch-shape. Likewise, cavity 340a can have an arch-shape. Such configurations can allow the body portion 340 (for example, a lower section of the body portion 340) to wrap around and/or conform to a shape of a chin of the user 1. This can reduce the amount the mask 300 sticks out in front of the user's face. This can also advantageously allow the cavity 340a, filter 360, holes 347a, vents 345a, and/or inlet/outlet 343 to be more aligned with a direction of gas exhaled from the user's nasal passages, thereby allowing such exhaled gas to efficiently exit the mask 300.

Filter 360 can comprise a corrugated structure and/or profile. Filter 360 can be an anti-microbial and/or anti-bacterial filter. Filter 360 can be configured to filter out particles from the air (e.g., dusts, mists, fumes, etc.) prior to inhalation by the user and/or can filter out particles in the user's breath before allowing such exhaled breath to exit an interior space defined by the mask 300 when in use. Filter 360 can be a disposable or reusable N95, N100, and/or HEPA type filter. In some implementations, filter 360 can filter out particulates having a size of 0.3 microns or larger. In some implementations, mask 300 (for example, body portion 340) is configured to allow filter 360 to be replaced.

FIGS. 3C-3D illustrate side and enlarged side views of mask 300 secured to the user 1 with certain portions of the mask 300 (and user's face) shown in dotted lines so as to better illustrate optional internal electronic components that can be integrated into mask 300. Mask 300 can include a battery 310 (which can be a coin cell battery), a circuit board and/or electronic module (that can include a circuit board) 350. In some implementations, mask 300 (for example, body portion 340) is configured to allow battery 310 to be removed and/or replaced, for example, via a removable component 311 that can hold and/or surround battery 310. Such component 311 can include an opening having a shape that corresponds to a perimeter or portion of the perimeter of batter 310 (which can have a circular shape). Mask 300 can include a processor, communication module, storage device, and/or information element that can be coupled and/or in communication with circuit board 350, and such processor, communication module, storage device, and/or information element can be similar or identical to processor 102, communication module 108, storage device 104, and/or information element 106 discussed above.

Mask 300 can include one or more physiological or other sensors that can be coupled and/or in communication with circuit board 350, for example, via cable(s). For example, mask 300 can include an oximetry sensor 312 coupled to circuit board 350 via cable 315a and/or temperature sensor 314b coupled to circuit board 350. Oximetry sensor 312 can be configured to be placed on the user's nose when mask 300 is in use. In some implementations, cable 315a mechanically couples oximetry sensor 312 with the body portion 340 and electrically couples oximetry sensor 312 with circuit board 350. Oximetry sensor 312 can be configured to secure to an outside of the user's nose. In some implementations, oximetry sensor 312 comprises a clip configured to secure around a nostril of the user, as shown in FIG. 3D. Alternatively, in some implementations, oximetry sensor 312 does not comprise a clip. Although the figures illustrate oximetry sensor 312 coupled with body portion 340 via cable 315a such that oximetry sensor 312 can be spaced from body portion 340 when mask 300 is in use, in some variants, oximetry sensor 312 is formed on and/or within body portion 340, for example, an upper section of body portion 340 such that oximetry sensor 312 is positioned adjacent skin of the nose when mask 300 is in use. Oximetry sensor 312 an include one or more emitters and one or more detectors for obtaining physiological information indicative of one or more blood parameters of the user 1. Oximetry sensor 312 can include such one or more emitters and one or more detectors in a transmissive arrangement (for example, where oximetry sensor 312 comprises a clip) or a reflective arrangement (for example, where body portion 340 operably positions oximetry sensor 312 adjacent skin of the nose). Oximetry sensor 312 can be utilized to determine one or more physiological parameters such as any of those discussed herein. Oximetry sensor 312 can be similar or identical in some or many respects to oximetry sensor 112 discussed above.

With continued reference to FIG. 3D, in some implementations, mask 300 includes a temperature sensor 314a and/or temperature sensor 314b. Temperature sensor 314a can be positioned on and/or within a portion of body portion 340, such as an upper section of body portion 340. Body portion 340 (for example, an upper section thereof) can operably position temperature sensor 314a adjacent skin on the user's nose (for example, on or near the bridge of the user's nose) when mask 300 is in use. In some implementations, temperature sensor 314a is coupled with circuit board 350 via a cable that can be positioned within the body portion 340. Temperature sensor 314a can advantageously be utilized for measuring skin temperature of the user 1.

Temperature sensor 314b can be operably positioned to facilitate measurement of temperature of exhaled gases of the user 1. For example, temperature sensor 314b can be operably positioned within the body portion 340 (or portions thereof) so as to be in an exit path of exhaled gases flowing and/or being directed through outlet 343 of mask 300. In some implementations, temperature sensor 314b can be positioned within cavity 340a and/or positioned on and/or secured to filter 360. FIGS. 3E-3H show implementations of a location of the temperature sensor 314b on filter 360. Temperature sensor 314b can be positioned on and/or secured to a top of the filter or a bottom of the filter 360 and/or can be positioned in various locations along a plane defined along a top or bottom of the filter 360. Such configuration advantageously allows the temperature sensor 314b to be used to measure temperature of exhaled gases from the user 1. Measuring exhaled breath temperature (“EBT”) can provide valuable insight as to a status of the user 1. For example, a rise in EBT may be indicative of a fever, infection (such as a viral respiratory infection), asthma, chronic obstructive pulmonary lung disease (“COPD”), among other things. As shown in FIG. 3D, temperature sensor 314b can be coupled with circuit board 350 via a cable 315b. In some implementations, mask 300 includes both temperature sensor 314a and temperature sensor 314b. Such implementations can allow skin temperature value(s) (obtained using temperature sensor 314a) to be compared and/or assessed in combination with EBT values (obtained using temperature sensor 314b). For example, in some implementations, a processor of mask 300 are configured to determine a status of the user 1 based on temperature value(s) obtained from both of temperature sensor 314a and temperature sensor 314b. In some implementations, temperature values are obtained using temperature sensor 314a after it is determined that temperature values obtained using temperature sensor 314b are above a certain threshold, or vice versa.

With reference to FIGS. 3A-3D, mask 300 can include a status indicator 330. Status indicator 330 can be positioned on a portion of body portion 340 and can be configured to indicate a status of the mask 300 and/or of the user 1 (for example, based on one or more physiological parameters determined from one or more sensors of mask 200). Status indicator 330 can be similar or identical to status indicator 130 discussed above. Status indicator 330 can be in communication with (for example, connected via a wire) circuit board 350.

Similar to as discussed with respect to mask 100, mask 300 can be configured to communicate with separate devices, for example, via a communication module of mask 300 that can be similar or identical to communication module 108. For example, mask 300 can be configured to communicate with one or more auricular devices 190 (shown in FIGS. 3A, and 3C) that can secure to the user's ear(s). In some implementations, mask 300 can be utilized in a system including auricular device(s) 190. Either or both of mask 300 and auricular device(s) 190 can include one or more physiological sensors usable to measure one or more physiological parameters of the user 1. In some implementations, mask 300 includes a microphone that can be similar or identical to microphone 126. In such implementations, such microphone can be used to transmit sounds from the user to the user's own ear via auricular device(s) 190 and/or to an auricular device 190 and/or other device of another user to facilitate audio communication therebetween. As mentioned previously, auricular device 190 can be similar or identical to any of the auricular devices described in U.S. Pat. Application No. 63/222,284, filed Jul. 15, 2021, and titled “Auricular Device,” which is hereby incorporated by reference in its entirety.

Face mask 300 can be configured to communicate with separate devices via a communication module of mask 300 that can be similar or identical to communication module 108. For example, mask 300 can be configured to wirelessly transmit processed and/or unprocessed obtained physiological information to a mobile phone which can include one or more processors configured to execute an application that generates a graphical user interface displaying information representative of the processed or unprocessed physiological and/or other information obtained from mask 300. This can advantageously allow a user 1 to monitor physiological information obtained from mask 300 during use via a separate device (for example, mobile phone). Such physiological information can include, for example, any of that which is described herein.

Face mask 300 and/or portions thereof can be configured to be washable and/or reusable. For example, in some implementations, electronic components of mask 300 are enclosed and/or sealed from water ingress such that portions of mask 300 can be wiped, and/or washed. As another example, in some implementations, inlet/outlet 343 can be flushed by allowing water to run through holes 347a, cavity 340a, and/or vents 345a. As another example, in some implementations, window 342 is configured to be washed when connected to body portion 340 and/or when removed therefrom (for example, in embodiments where window 343 is configured to be removed from body portion 340). In some implementations, straps 346a, 346b can be washed. Such configurations can advantageously allow mask 300 to be cleaned and/or sanitized, thereby extending service life of the mask 300.

FIG. 3I illustrates an alternative embodiment of a face mask 300′ that can be identical to face mask 300 except with respect to filter 360′ and cavity 340a′ as discussed below. Instead of filter 360, face mask 300′ includes a filter 360′ (which can also be referred to as a “filter assembly”). Additionally, face mask 300′ includes cavity 340a′ which can have a different shape and/or configuration than cavity 340a. Filter 360′ can include a number of portions that are separable and/or removable from one another, as explained below. Cavity 340a′ can have a smaller height than cavity 340a (see FIG. 3D and FIG. 3I). In some implementations, a temperature sensor (such as temperature sensor 314b) can be positioned atop and/or secured to filter assembly 360′ which can enable EBT to be determined similar to as discussed elsewhere herein.

FIGS. 3J-3L illustrate various views of filter assembly 360′. Filter assembly 360′ can include a first frame 360a′, a second frame 360b′, a wicking element 360c′, and a filter element 360d′. FIG. 3J illustrates an exploded view of filter assembly 360′. FIG. 3K illustrates a partial cross-section through filter assembly 360′ in an assembled configuration and FIG. 3L illustrates a top perspective view of filter assembly 360′ in an assembled configuration. In some implementations, filter assembly 360′ is positioned within cavity 340a′ such that frame 360b′ is a bottommost portion of the filter assembly 360′ (for example, frame 360b′ is positioned vertically below filter element 360d′, wicking element 360c′, and frame 360a′). In such configuration, frame 360a′ can be positioned at a topmost portion of the filter assembly 360′. In such configuration, exhaled gas from the user's nose and/or mouth can flow downward through the filter assembly 360′, passing through frame 360a′, wicking element 360c′, filter element 360d′, and frame 360b′, for example, in such order. Additionally, air flowing into the interior space of the mask 300 (for example, during inhalation by the user 1) can flow upward through the filter assembly 360′, passing through frame 360b′, filter element 360d′, wicking element 360c′, and frame 360a′, for example, in such order. Frames 360a′, 360b′ can be removably connected together or permanently secured to one another so as to sandwich wicking element 360c′ and/or filter element 360d′ together and/or therebetween. Filter assembly 360′ and/or any of frame 360a′, wicking element 360c′, filter element 360d′, and frame 360b′ can be disposable, reusable, and/or replaceable.

Wicking element 360c′ can be sized and/or shaped to wick moisture from exhaled gases to an outer portion thereof (for example, to and/or toward an outer perimeter of wicking element 360c′. In some implementations, wicking element 360c′ includes an outer ring-shaped recess having a triangular cross-section, as shown. Filter element 360d′ can be an anti-microbial and/or anti-bacterial filter. Filter element 360d′ can be configured to filter out particles from the air (e.g., dusts, mists, fumes, etc.) prior to inhalation by the user 1 and/or can filter out particles in the user's breath before allowing such exhaled breath to exit an interior space defined by the mask 300 when in use. Filter element 360d′ can be a disposable or reusable N95, N100, and/or HEPA type filter. In some implementations, filter element 360d′ can filter out particulates having a size of 0.3 microns or larger. Filter element 360d′ can comprise a corrugated structure, which can in some cases increase surface area for increased filtering efficiency and/or breathability. In some implementations, filter element 360d′ is configured to prevent viruses from passing therethrough into and/or out of the interior space of mask 300.

Although FIGS. 3J-3L illustrate filter assembly 360′ having a circular shape, filter assembly 360′ can have an alternative shape. For example, filter assembly 360′ can have a circular shape, among others. Cavity 340a′ can have a shape that corresponds to a shape of the filter assembly 360′. In some implementations, filter assembly 360′ and cavity 340a′ have an arch shape like that shown and described with reference to filter 360 and cavity 340a above which can allow provide conformity with and/or around the user's chin.

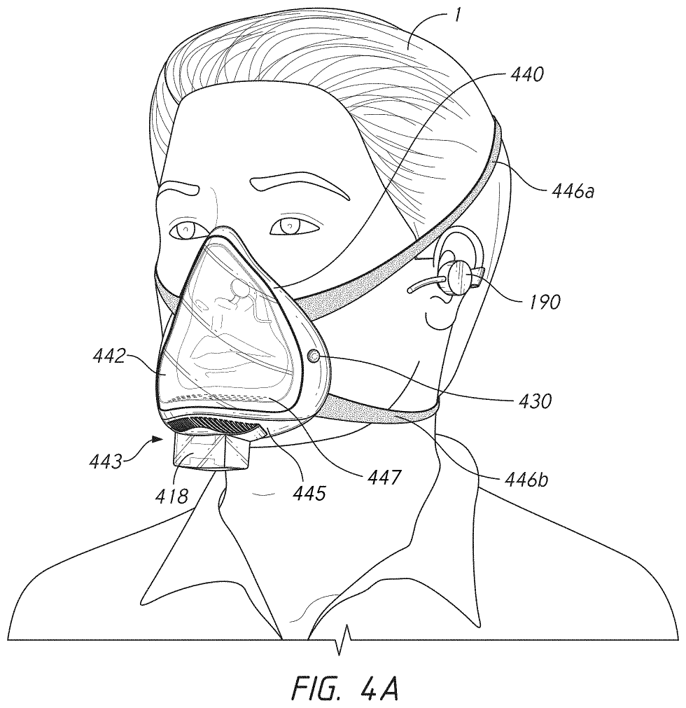

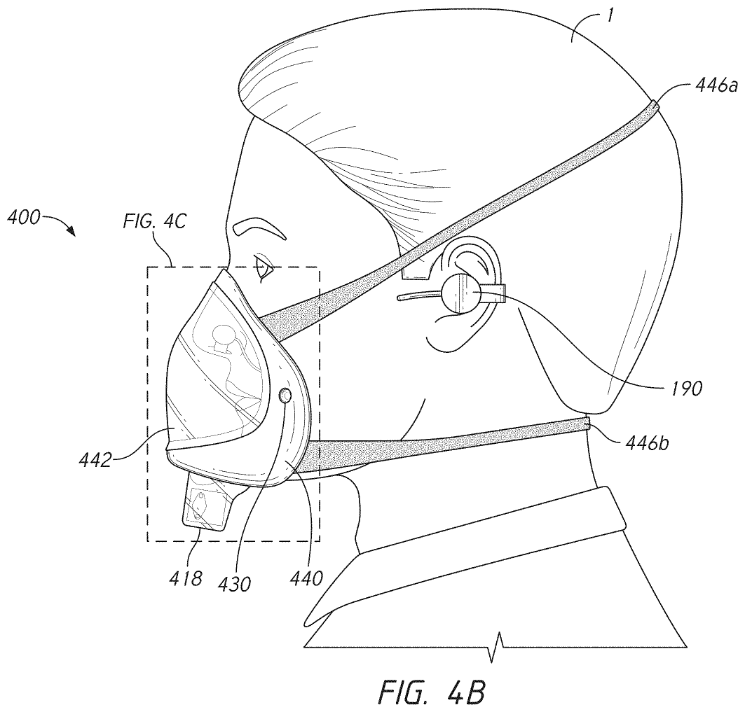

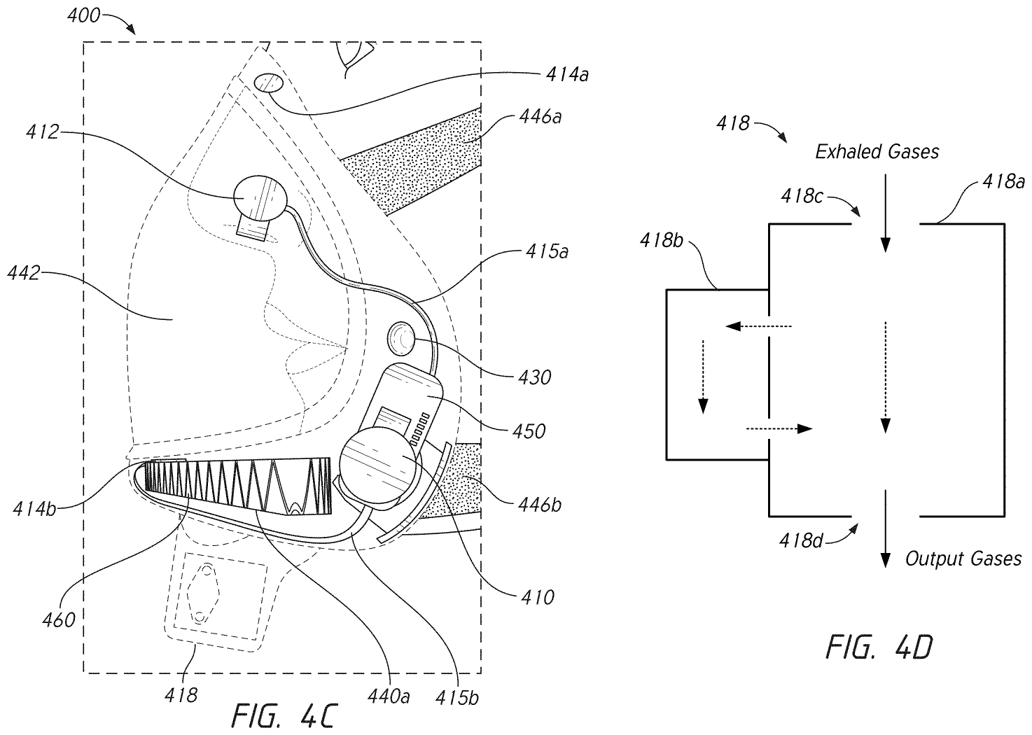

FIGS. 4A-4C illustrate an embodiment of a face mask 400 that can be identical to face mask 300 in some or many respects. FIGS. 4A-4C illustrate a face mask 400 and a capnograph module 418. Face mask 400 can include a body portion 440, window 442, status indicator 430, straps 446a, 446b, and/or inlet/outlet 443 which can be identical to body portion 340, window 342, status indicator 330, straps 346a, 346b, and/or inlet/outlet 343 (respectively). Additionally, face mask 400 can interact with one or more auricular devices 190 in a similar or identical manner as that described above with respect to face mask 300. FIG. 4C illustrates an enlarged side view of mask 400 with external portions of mask 400 and portions of the user's face shown in dotted lines similar to that shown in FIG. 3D. Mask 400 can include an oximetry sensor 412, circuit board 450, cables 415a, 415b, battery 410, temperature sensor 414a, temperature sensor 414b, filter 460, cavity 440a, which can be similar or identical to oximetry sensor 312, circuit board 350, cables 315a, 315b, battery 310, temperature sensor 314a, temperature sensor 314b, filter 360, and cavity 340a (respectively).

Capnograph module 418 can be and/or comprise structure that is removably or permanently attached to body portion 440 (for example, a lower section of body portion 440) at and/or around an outlet 443 of mask 300 (which can be similar or identical to outlet 343). In some implementations, body portion 440 (for example, a lower section of body portion 440) comprises structure that allows capnograph module 418 to removably secure (for example, via a snap fit) to body portion 440 underneath outlet 443. For example, in some implementations, capnograph module 418 includes one or more protrusions that can secure within one or more recesses on a bottom wall or surface 445 of body portion 440 (for example, that can be identical to bottom wall or surface 345). Additionally or alternatively, such bottom wall or surface 445 of body portion 440 can include one or more protrusions that can secure within one or more recesses on capnograph module 418. In some implementations, capnograph module 418 is integral with body portion 440. For example, in some implementations, capnograph module 418 is contiguous with and extends downward from lower section of body portion 440 in front of a chin of user 1 when mask 400 is in use.

In some implementations, capnograph module 418 covers an entirety of outlet 443. In some implementations, capnograph module 418 does not cover an entirety of outlet 443. Outlet 443 (which can also be an inlet of mask 400 as explained with reference to other masks described herein) can comprise one or more or a plurality of openings, which can be holes and/or vents similar or identical to that described above with reference to mask 300. Body portion 440 can include bottom wall or surface 445 and/or inner wall or surface 447. In some implementations, bottom wall or surface 445 includes a plurality of vents that can be identical to vents 345a and/or inner wall or surface 447 includes a plurality of holes that can be identical to holes 347a (see FIG. 3B). In some implementations, bottom wall or surface 445 includes a single opening instead of such plurality of vents and/or inner wall or surface 347 includes a single opening instead of such plurality of holes. In some implementations, such single opening on each of the bottom wall 445 and the inner wall 447 are aligned (for example, vertically). In some implementations, capnograph module 418 covers an entirety of such single opening on bottom wall or surface 445. Alternatively, in some implementations, capnograph module 418 does not cover such a single opening on bottom wall or surface 445, which can allow for some exhaled gases to exit outlet 443 without passing through capnograph module 418. In some implementations, mask 400 is configured such that an inlet of mask 400 is separate from an outlet of mask 400 that is coupled with capnograph module 418. In some implementations, an inlet passageway defined by mask 400 includes a filter, such as filter 460, and an outlet passageway defined by mask 400 does not include a filter so that properties of exhaled gases can be measured by capnograph module 418 without being subjected to filtration by a filter. As another example, in some implementations, cavity 440a can be partitioned between a first portion that at least partially defines an inlet passageway for inhalation by user 1 and an outlet passageway that at least partially defines an outlet passageway for exhaled gases of user 1, where such outlet passageway does not include a filter so as to not alter the composition of exhaled gases prior to delivery to capnograph module 418.

Capnograph module 418 can advantageously be utilized to measure physiological parameters by analyzing gases exhaled by user 1 through outlet 443. Such physiological parameters can be, for example, end-tidal respiratory gases including oxygen (O₂), carbon dioxide (CO₂), and nitrous oxide (N₂O), among others, as well as respiratory rate. Capnograph module 418 can be beneficial for patients in clinical environments and persons during daily activities inside and/or outdoors. Capnograph module 418 can also be advantageously be utilized by athletes where fitness levels and performance capacity is crucial. In some implementations, capnograph module 418 is configured to determine maximal oxygen uptake (VO₂ max), a measurement of the maximum amount of oxygen a person can utilize during intense exercise indicative of aerobic endurance before and/or during training. Capnograph module 418 can also be advantageously be utilized by law enforcement, military personnel, and/or firefighters all of who may be subjected to settings and environments having compromised air quality and/or airborne toxins.

In some implementations, capnograph module 418 can advantageously be utilized to not only determine physiological parameter(s) in exhaled gases, but also to generate an alert based on such determined physiological parameter(s). For example, a processor of capnograph module 418 and/or a processor of mask 400 can determine physiological parameter(s) based on analyzed exhaled gases, compare such physiological parameter(s) to threshold(s), and initiate and/or transmit an alert based on such comparison. In some implementations, such alert can be displayed via a portion of mask 400, for example, via status indicator 430. In some implementations, such alert can be transmitted to a separate computing device, for example, a mobile phone, or, in the case of law enforcement, military personnel, or firefighters, a monitoring station tasked with monitoring status(es) of associated personnel. By way of non-limiting example, where mask 400 is worn by a firefighter during a fire incident, capnograph module 418 can be configured to generate an alert indicative of an amount of airborne particulates associated with fire smoke. Such alert can allow an individual firefighter and/or a monitoring station to know when a fainting or other event may take place, which can allow action to be immediately taken to prevent such event.

Capnograph module 418 can comprise structure that is similar or identical to any of the capnography measurement systems (or portions thereof) described in U.S. Pat. No. 10,532,174, titled “Assistive Capnography Device,” which is incorporated by reference herein in its entirety. Capnograph module 418 can be configured to operate in a similar or identical manner to any of the capnography measurement systems (or portions thereof) described in U.S. Pat. No. 10,532,174. FIG. 4D which illustrates an exemplary schematic diagram of certain features that can be included in capnograph module 418. Capnograph module 418 can include a housing that can include and/or define an airway channel 418a (which may be referred to as a “main” or “primary” air way or “flow” channel) that is in fluid communication with outlet 443 of mask 400 so as to allow gases exhaled by the user 1 through outlet 443 to flow within channel 418a. Capnograph module 418 (for example, chamber 418a and/or a housing that includes chamber 418b) can include an inlet 418c and outlet 418d.

In some implementations, capnograph module 418 includes a measuring chamber 418b in fluid communication with channel 418a. Capnograph module 418 can be configure to direct and/or guide a portion of the exhaled gases flowing through the channel 418a to measuring chamber 418b for sampling. Capnograph module 418 can be configured to measure one or more physiological parameters by analyzing respiratory gases in measuring chamber 418b. For example, capnograph module 418 can include a measurement head that can be configured to operate in a similar or identical manner as any of the measurement heads described in U.S. Pat. No. 10,532,174. Capnograph module 418 can include one or more emitters (such as LEDs) configured to emit light of one or more wavelengths into measuring chamber 418b (which can include sampled exhaled respiratory gases) and one or more sensors configured to detect at least a portion of the emitted light after passing through gas within chamber 418b. Such one or more emitters can be infrared emitters and/or such one or more sensors can be infrared detectors. Light from the emitter(s) can pass through gas within chamber 418b and, depending on characteristics of such gas, can be partially absorbed. Such partially absorbed light can be detected by the sensor(s) and intensity of the detected light can be determined, for example, by a processor of capnograph module 418 and/or by a processor of mask 400. Such sensor(s) can be similar or identical to any of those described in U.S. Pat. No. 10,532,174. Measuring the intensity of the light that was not absorbed into the gas within chamber 418b can facilitate determination of a quantification of concentration of a gas or gases within chamber 418b. This can in turn facilitate identification of which gases and/or agents are present in gases exhaled by user 1. In some implementations, capnograph module 418 includes an optical filter (such as a narrow band optical filter) that can at least partially filter light after passing through gas within chamber 418b and prior to being detected by the one or more sensors. Any or all of the emitter(s), sensor(s), and/or optical filter can be positioned adjacent or within chamber 418b.

Capnograph module 418 can include a processor generally including circuitry that can process one or more signals generated and/or transmitted by the sensor(s) of capnograph module 418 responsive to detected light. Such processor can be similar or identical to that described in U.S. Pat. No. 10,532,174. In some implementations, for example, where capnograph module 418 is removably connectable to mask 400, capnograph module 418 includes a separate processor than a processor of mask 400. In such implementations, such separate processor of capnograph module 418 can be configured to determine one or more physiological parameters based on exhaled gases and can communicate (via wired or wireless means) such physiological parameters to a processor of mask 400. Mask 400 can, for example, then communicate such physiological parameters and/or information related to such physiological parameters to a separate computing device (for example, a mobile phone). In some implementations, for example, where capnograph module 418 is integral with body portion 440, one or more sensors of capnograph module 418 can be coupled with a processor of mask 400 and can transmit signal(s) responsive to detected light to such processor of mask 400 which can then determine one or more physiological parameters.

As shown in FIG. 4D, capnograph module 418 (for example, chamber 418a and/or a housing that includes chamber 418b) can include an outlet 418d (for example, an opening) that can allow exhaled gas to exit the capnograph module 418. In some implementations, such outlet 418d of capnograph module 418 is configured to direct exhaled gases downward when in use. For example, where capnograph module 418 is integral or removably coupled with a lower section of body portion 440 of mask 400 at or near a chin of user 1 when in use, outlet 418d can be operably positioned to direct exhaled gas downward toward the user's feet and/or the ground. This can advantageously minimize the potential for re-breathing of exhaled gases. This can also advantageously inhibit the tendency that exhaled gases flow in front of the mask 400, thereby minimizing fogging of mask 400 and/or interference with the user's line of sight.

In some implementations, capnograph module 418 can be configured to removably connect to a ventilator, for example, via a tube. For example, in some implementations, capnograph module 418 includes a housing (which can include chamber 418a) having an outlet 418d that is configured to removably connect (for example, via a snap fit connection) to a tube and/or connector. Such configurations can advantageously allow for quick connection of an oxygen delivery source when or if oxygen levels of the user 1 are detected to be below a threshold by capnograph module 418 and/or by mask 400.

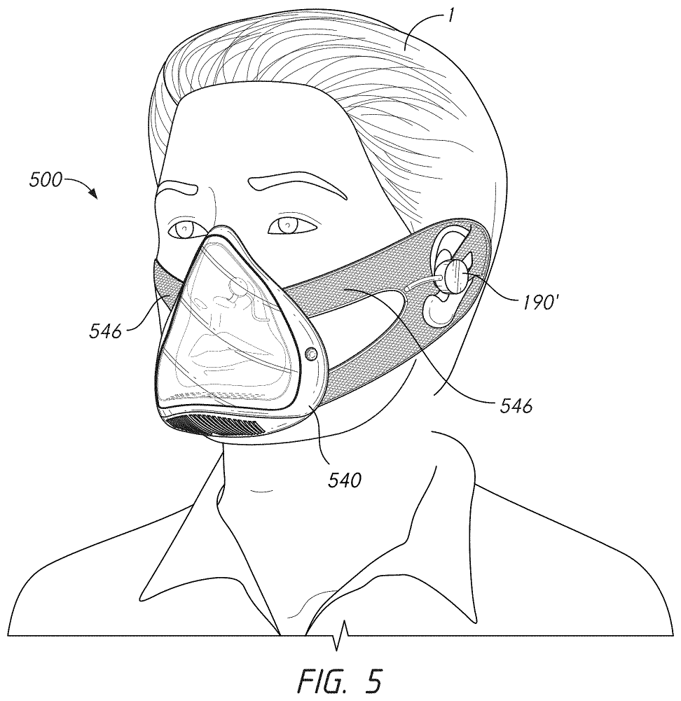

FIG. 5 illustrates a face mask 500 that includes a strap 546 coupled to a body portion 540. Strap 546 can be integral with and/or removably connectable to an auricular device 190′. Auricular device 190′ can be similar or identical to auricular device 190 discussed above. Advantageously, such configuration can allow auricular device 190′ to be positioned and/or positionable at the user's ear when face mask 500 is secured to the user 1. Strap 546 can be configured to extend and/or secure around an ear of user 1. Mask 500 can include two straps 546 which oppose each other and secure around ears of user 1. In some implementations, straps 546 do not wrap around the user's head. Such straps 546 can comprise a material similar to any of that described elsewhere herein with respect to other straps. In some implementations, each of the straps 546 comprises two arms extending outward from body portion 540 defining an opening therebetween, as shown in FIG. 5. In some implementations, mask 500 includes two straps 546 extending to and around each ear but only one auricular device 190′ coupled to one of the two straps 546. Any of the masks described herein can employ straps 546 and auricular device 190′ integral with and/or removably connectable to strap 546.

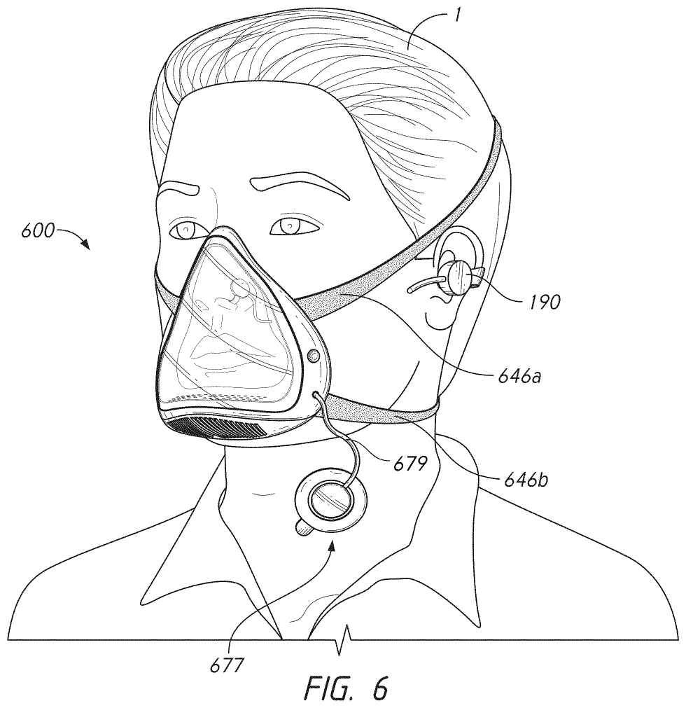

FIG. 6 illustrates a face mask 600 that includes an acoustic sensor 677. Mask 600 can be similar or identical to mask 300 described above in some or many respects. Acoustic sensor 677 can be coupled to a circuit board and/or electronic module that can include a circuit board which can be similar or identical to circuit board 350. Acoustic sensor 677 can be coupled to such circuit board and/or electronic module of mask 600 via a cable 679. As shown, acoustic sensor 677 can be secured to a neck of user 1 when face mask 600 is in use. For example, acoustic sensor 677 can be placed at or near the carotid artery. Acoustic sensor 677 can comprise an adhesive material that allows adhesive securement to the user's skin.

Acoustic sensor 677 (which can also be referred to as an “acoustic respiratory sensor” or “respiratory sensor”) can comprise an acoustic transducer, such as a piezoelectric element. Acoustic sensor 677 can detect respiratory and other biological sounds of a patient and provide signals reflecting these sounds to a processor of mask 600 (such as any of those discussed herein). Acoustic sensor 677 can be a piezoelectric sensor or the like that obtains physiological information reflective of one or more respiratory parameters of the user 1. These parameters can include, for example, respiratory rate, inspiratory time, expiratory time, inspiration-to-expiration ratio, inspiratory flow, expiratory flow, tidal volume, minute volume, apnea duration, breath sounds, rales, rhonchi, stridor, and changes in breath sounds such as decreased volume or change in airflow. In addition, in some cases the acoustic sensor 677, or another lead of the acoustic sensor 677 (not shown), can measure other physiological sounds such as heart rate (e.g., to help with probe-off detection), heart sounds (for example, S1, S2, S3, S4, and murmurs), and changes in heart sounds such as normal to murmur or split heart sounds indicating fluid overload. In some implementations, a second acoustic sensor (which can be similar to acoustic sensor 677) can be utilized alongside acoustic sensor 677 over the chest of the user 1 for additional heart sound detection. Acoustic sensor 677 can be used to generate an exciter waveform that can be detected by an optical sensor of mask 600 (that can be similar or identical to any of the optical sensors discussed herein) and/or an optical sensor that is placed on a fingertip of the user 1. The velocity of the exciter waveform can be calculated by a processor in the mask 600. From this velocity, such processor can derive a blood pressure measurement or blood pressure estimate. The processor can output the blood pressure measurement for display.

Any of the face masks disclosed herein (for example, mask 100, 200, 300, 400, 500, 600) can include a fan for providing active ventilation. Alternatively, some implementations of masks 100, 200, 300, 400, 500, 600 do not include a fan. Any of the face masks disclosed herein (for example, mask 100, 200, 300, 400, 500, 600) can be utilized with any of the systems, methods, and/or devices for contact tracing and/or other purposes which are disclosed in U.S. application Ser. No. 17/206,794, filed Mar. 19, 2021, and titled “Health Monitoring System for Limiting the Spread of an Infection in an Organization,” which is hereby incorporated by reference in its entirety. Any of the face masks disclosed herein (for example, mask 100, 200, 300, 400, 500, 600) can be utilized with any of the systems, methods, and/or devices disclosed in U.S. application Ser. No. 17/207,469, filed Mar. 19, 2021, and titled “Remote Patient Management and Monitoring Systems and Methods,” which is hereby incorporated by reference in its entirety. For example, any of the face masks disclosed here (for example, mask 100, 200, 300, 400, 500, 600) can be capable of wirelessly transmitting data (for example, physiological data) to a mobile computing device such as iOS or Android™ enabled mobile phones via a wireless link which can communicate with a remote patient management system as described in U.S. application Ser. No. 17/207,469.

### Additional Considerations and Terminology

Although this invention has been disclosed in the context of certain preferred embodiments, it should be understood that certain advantages, features and aspects of the systems, devices, and methods may be realized in a variety of other embodiments. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred embodiments.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain embodiments and examples have been described herein, it will be understood by those skilled in the art that many aspects of the systems and devices shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (for example, not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. A face mask configured to secure to a face of a user, provide filtration of air prior to inhalation by the user, and measure one or more physiological parameters of the user, the face mask comprising:
a body portion configured to be secured to the user's face and cover a mouth and nasal passages of the user, the body portion configured to at least partially define an interior space when secured to the user's face, the body portion comprising:
an upper section configured to be positioned around at least a portion of a nose of the user and conform to at least a portion of a shape of the user's nose when the body portion is secured to the user's face;
a lower section configured to be positioned proximate a chin of the user when the body portion is secured to the user's face;
an inlet configured to allow air to flow into said interior space during inhalation by the user;
an outlet configured to allow exhaled gases from the user to flow outside said interior space, wherein said inlet and outlet are located in the lower section of the body portion and are configured to face downward when the body portion is secured to the user's face;
a filter positioned adjacent the inlet and the outlet, wherein the filter is configured to filter out particles in said air prior to inhalation by the user; and
at least one strap connected to the body portion and configured to secure the body portion to the user;
a power source;
one or more hardware processors; and
an oximetry sensor in communication with said one or more hardware processors and configured to be positioned at the user's nose when the face mask is in use, wherein the oximetry sensor comprises at least one emitter configured to emit one or more wavelengths into tissue of the user's nose and at least one detector configured to detect at least a portion of the emitted light after passing through at least a portion of said tissue, said at least one detector configured to transmit one or more signals to said one or more hardware processors responsive to detected light;
wherein said one or more hardware processors are configured to determine said one or more physiological parameters based on said one or more signals transmitted by said at least one detector.

2. The face mask of claim 1, wherein said one or more physiological parameters comprises at least one of oxygen saturation and pulse rate.

3. (canceled)

4. (canceled)

5. (canceled)

6. (canceled)

7. The face mask of claim 1, wherein the oximetry sensor is secured to the upper section of the body portion such that, when the upper section is positioned around the at least the portion of the nose of the user, the oximetry sensor is positioned adjacent skin of the nose and the at least one emitter and the at least one detector are arranged in a reflectance arrangement with respect to the skin.

8. (canceled)

9. (canceled)

10. (canceled)

11. (canceled)

12. (canceled)

13. (canceled)

14. (canceled)

15. The face mask of claim 1, further comprising a temperature sensor operably positioned by the upper section of the body portion such that, when the upper section is positioned around the at least the portion of the nose of the user, the temperature sensor is positioned adjacent skin of the nose.

16. The face mask of claim 1, wherein said inlet and said outlet occupy the same space in the body portion.

17. (canceled)

18. The face mask of claim 1, further comprising a status indicator configured to indicate at least one of a status of the face mask and a status of the user.

19. The face mask of claim 18, wherein said status indicator comprises one or more light sources.

20. The face mask of claim 19, wherein said one or more hardware processors are configured to alter a characteristic of said one or more light sources based on said determined one or more physiological parameters.

21. The face mask of claim 20, wherein said one or more hardware processors are configured to alter said characteristic of said one or more light sources based on a comparison of said determined one or more physiological parameters to one or more thresholds.

22. (canceled)

23. (canceled)

24. The face mask of claim 1, wherein said face mask is configured to wirelessly transmit said determined one or more physiological parameters to a mobile computing device.

25. (canceled)

26. A system comprising the face mask of claim 24 and a mobile software application configured to execution by one or more hardware processors of said mobile computing device, wherein the mobile software application is configured to execute commands to enable the mobile computing device to:
wirelessly receive said determined one or more physiological parameters;
generate a graphical user interface on a display of the mobile computing device; and
display, in at least a portion of the graphical user interface, at least one of said determined one or more physiological parameters and information related to said determined one or more physiological parameters.

27. (canceled)

28. (canceled)

29. A face mask configured to secure to a face of a user and measure one or more physiological parameters of the user, the face mask comprising:
a body portion configured to be secured to the user's face and cover a mouth and nasal passages of the user;
at least one strap connected to the body portion and configured to secure the body portion to the user;
a power source;
one or more hardware processors; and
an oximetry sensor in communication with said one or more hardware processors and configured to be positioned at the user's nose when face mask is in use, wherein the oximetry sensor comprises at least one emitter configured to emit one or more wavelengths into tissue of the user's nose and at least one detector configured to detect at least a portion of the emitted light after passing through at least a portion of said tissue, said at least one detector configured to transmit one or more signals to said one or more hardware processors responsive to detected light;
wherein said one or more hardware processors are configured to determine said one or more physiological parameters based on said one or more signals transmitted by said at least one detector.

30. The face mask of claim 29, wherein the body portion is configured to at least partially define an interior space when secured to the user's face, and wherein the body portion comprises:
an upper section configured to be positioned around at least a portion of a nose of the user and conform to at least a portion of a shape of the user's nose when the body portion is secured to the user's face;
a lower section configured to be positioned near a chin of the user when the body portion is secured to the user's face;
an inlet configured to allow air to flow into said interior space during inhalation by the user;
an outlet configured to allow exhaled gases from the user to flow outside said interior space; and
a filter positioned adjacent the inlet and the outlet, wherein the filter is configured to filter out particles in said air prior to inhalation by the user.

31. The face mask of claim 30, wherein said inlet and outlet are located in the lower section and are configured to face downward when the body portion is secured to the user's face.

32. The face mask of claim 30, wherein said inlet and said outlet occupy the same space in the body portion.

33. The face mask of claim 30, wherein the lower section of said body portion comprises:
an outer wall that faces downward when the body portion is secured to the user's face;
an inner wall spaced above the outer wall; and
a cavity positioned between the outer and inner walls, where said filter is positioned within said cavity.

34. The face mask of claim 33, further comprising a first plurality of openings in said outer wall and a second plurality of openings in said inner wall, wherein said inlet and said outlet are at least partially defined by said first and second plurality of openings.

35. The face mask of claim 34, wherein each of said first plurality of openings comprises a vent having a linear shape and wherein each of said second plurality of openings comprises a hole having circular shape.

36. The face mask of claim 30, wherein the oximetry sensor is operably positioned such that, when the upper section is positioned around the at least the portion of the nose of the user, the oximetry sensor is positioned adjacent skin of the nose and the at least one emitter and the at least one detector are arranged in a reflectance arrangement with respect to the skin.

37. The face mask of claim 30, further comprising a temperature sensor operably positioned such that, when the upper section is positioned around the at least the portion of the nose of the user, the temperature sensor is positioned adjacent skin of the nose.

38-112. (canceled)
